(19) United States
(12) Patent Application Publication
(10) Pub. No.: US 2010/0099714 A1
(43) Pub. Date: Apr. 22, 2010

(54) AMPA and NMDA Receptor Antagonists for Neurodegenerative Diseases

(75) Inventors: Hiroshi Yoshino, Ibaraki (JP); Takahisa Hanada, Ibaraki (JP); Shigeru Akasofu, Ibaraki (JP)

Correspondence Address: CLARK & ELBING LLP, 101 FEDERAL STREET, BOSTON, MA, 02110, US

(73) Assignee: Eisai R&D Management Co., Ltd., Tokyo (JP)

(21) Appl. No.: 12/528,377

(22) PCT Filed: Mar. 5, 2008
(86) PCT No.: PCT/JP2008/054422

§ 371 Date: Aug. 24, 2009

Related U.S. Application Data
(60) Provisional application No. 60/904,781, filed on Mar. 5, 2007.

(51) Int. Cl. A61K 31/444; A61P 25/00; A61P 9/00; A61P 27/02; A61P 29/00

(52) U.S. Cl. 514/334

(57) ABSTRACT

The invention provides methods for treating, preventing, and delaying the onset of neurodegenerative diseases (e.g., Alzheimer's disease, Parkinson's disease, Huntington's disease, amyotrophic lateral sclerosis, multiple sclerosis, Pick's disease, and the like), dementia, mild cognitive impairments, glaucoma, ocular hypertension, and pain (e.g., neuropathic pain, headaches, and the like) by administering therapeutically effective amounts of AMPA receptor antagonists and NMDA receptor antagonists to patents in need thereof. The invention also provides combinations, kits, and pharmaceutical compositions comprising therapeutically effective amounts of AMPA receptor antagonists and NMDA receptor antagonists. Exemplary AMPA receptor antagonists include 1,2-dihydropyridine compounds, such as 3-(2-cyanophenyl)-5-(2-pyridyl)-1-phenyl-1,2-dihydropyridin-2-one. Exemplary NMDA receptor antagonists include adamantane compounds, such as memantine.

### RELATED APPLICATION

This application claims priority under 35 U.S.C. §119 to U.S. Provisional Application No. 60/904,781 filed Mar. 5, 2007, the disclosures of which are incorporated by reference herein in their entirety.

### FIELD OF THE INVENTION

The invention provides pharmaceutical compositions, combinations, and kits comprising AMPA receptor antagonists and NMDA receptor antagonists, and methods for treating diseases using AMPA receptor antagonists and NMDA receptor antagonists.

### BACKGROUND OF THE INVENTION

Cholinesterase inhibitors have conventionally been used to treat dementia, such as Alzheimer's Disease and Senile Dementia of the Alzheimer's Type. Other drugs including N-methyl-D-aspartic acid (NMDA) receptor antagonists are also used. Nonetheless, novel drugs and treatment methods are needed.

Memantine hydrochloride (NAMENDA®, Forest Pharmaceuticals, Inc.; AXURA®, Merz Pharmaceuticals), which is an NMDA receptor antagonist, is an amantadine derivative and is known to protect nerve cells and improves the symptoms of Parkinson's disease. Recently developed as a drug for treating moderate to severe Alzheimer's disease, memantine hydrochloride is provided in a liquid form or as film-coated tablets.

1,2-dihydropyridine compounds are known as α-amino-3-hydroxy-5-methyl-4-isoxazole propionic acid (AMPA) receptor antagonists and are described in U.S. Pat. No. 6,949,571. Methods for treating diseases and administering these compounds in conjunction with a cholinesterase inhibitor are described in WO 2006/107859.

There is a need in the art for treating neurodegenerative diseases and dementia using novel pharmaceutical compositions or combinations. The invention is directed to these, as well as other, important goals.

### SUMMARY OF THE INVENTION

The invention provides methods for the treatment and/or prophylaxis of Alzheimer's disease in a patient in need thereof by administering (i) at least one AMPA receptor antagonist and (ii) at least one NMDA receptor antagonist. The invention provides methods for delaying the onset of Alzheimer's disease by administering (i) at least one AMPA receptor antagonist and (ii) at least one NMDA receptor antagonist. It has been unexpectedly discovered that the combination of the AMPA receptor antagonist and the NMDA receptor antagonist produces synergistic effects in the treatment and/or prophylaxis of Alzheimer's disease, and in delaying the onset of Alzheimer's disease.

The invention provides methods for the treatment and/or prophylaxis of neurodegenerative diseases in a patient in need thereof by administering (i) at least one AMPA receptor antagonist and (ii) at least one NMDA receptor antagonist. The invention also provides methods for delaying the onset of neurodegenerative diseases by administering (i) at least one AMPA receptor antagonist and (ii) at least one NMDA receptor antagonist. It has been unexpectedly discovered that the combination of the AMPA receptor antagonist and the NMDA receptor antagonist produces synergistic effects in the treatment and/or prophylaxis of neurodegenerative diseases, and in delaying the onset of neurodegenerative diseases.

The invention provides methods for the treatment and/or prophylaxis of dementia or mild cognitive impairments in a patient in need thereof by administering (i) at least one AMPA receptor antagonist and (ii) at least one NMDA receptor antagonist. The invention provides methods for delaying the onset of dementia or mild cognitive impairments by administering (i) at least one AMPA receptor antagonist and (ii) at least one NMDA receptor antagonist. It has been unexpectedly discovered that the combination of the AMPA receptor antagonist and the NMDA receptor antagonist produces synergistic effects in the treatment and/or prophylaxis of dementia or mild cognitive impairments, and in delaying the onset of dementia or mild cognitive impairments.

The invention provides methods for the treatment and/or prophylaxis of glaucoma or ocular hypertension in a patient in need thereof by administering (i) at least one AMPA receptor antagonist and (ii) at least one NMDA receptor antagonist. The invention provides methods for delaying the onset of glaucoma or ocular hypertension by administering (i) at least one AMPA receptor antagonist and (ii) at least one NMDA receptor antagonist. It has been unexpectedly discovered that the combination of the AMPA receptor antagonist and the NMDA receptor antagonist produces synergistic effects in the treatment and/or prophylaxis of glaucoma or ocular hypertension, and in delaying the onset of glaucoma or ocular hypertension. In one embodiment, the active ingredients may be topically administered to the affected eye in the form of a gel or liquid (e.g., solution, suspension, and the like).

The invention provides methods for the treatment and/or prophylaxis of pain in a patient in need thereof by administering (i) at least one AMPA receptor antagonist and (ii) at least one NMDA receptor antagonist. The invention provides methods for delaying the onset of pain by administering (i) at least one AMPA receptor antagonist and (ii) at least one NMDA receptor antagonist. It has been unexpectedly discovered that the combination of the AMPA receptor antagonist and the NMDA receptor antagonist produces synergistic effects in the treatment and/or prophylaxis of pain, and in delaying the onset of pain. In one embodiment, “pain” refers to neuropathic pain. In another embodiment, “pain” refers to headaches, which includes migraines.

The invention provides pharmaceutical compositions comprising (i) at least one AMPA receptor antagonist; (ii) at least one NMDA receptor antagonist; and (iii) one or more pharmaceutically acceptable carriers.

The invention provides combinations comprising (i) at least one AMPA receptor antagonist and (ii) at least NMDA receptor antagonist. The combination may comprise two formulations that may be separately administered (e.g., simultaneously, sequentially) to a patient.

The invention provides kits comprising (i) at least one AMPA receptor antagonist; (ii) at least NMDA receptor antagonist; and (iii) instructions for simultaneous, separate or sequential use thereof for the treatment and/or prophylaxis of Alzheimer's disease, a neurodegenerative disease, or dementia, or for delaying the onset of Alzheimer's disease, a neurodegenerative disease, or dementia. The AMPA receptor antagonist and the NMDA receptor antagonist can be in the form of a composition or a combination.

The present invention relates to the following.

(1) A pharmaceutical composition comprising:

(i) perampanel, a pharmaceutically acceptable salt thereof, a hydrate thereof, or a hydrate of a pharmaceutically acceptable salt thereof.

(ii) memantine or a pharmaceutically acceptable salt thereof; and

(iii) one or more pharmaceutically acceptable carriers.

(2) The pharmaceutical composition of (1), wherein the pharmaceutical composition is used for treating (a) a neurodegenerative disease; (b) dementia; (c) a mild cognitive impairment; (d) glaucoma; (e) ocular hypertension; or (f) pain.

(3) A combination comprising:

(i) perampanel, a pharmaceutically acceptable salt thereof, a hydrate thereof, or a hydrate of a pharmaceutically acceptable salt thereof; and

(ii) memantine or a pharmaceutically acceptable salt thereof.

(4) The combination of (3), wherein the combination is administered separately to the patient or is administered to the patient in the form of a pharmaceutical composition.

(5) The combination of (3), wherein the combination is used for treating (a) a neurodegenerative disease; (b) dementia; (c) a mild cognitive impairment; (d) glaucoma; (e) ocular hypertension; or (f) pain.

(6) A kit comprising:

(i) perampanel, a pharmaceutically acceptable salt thereof, a hydrate thereof, or a hydrate of a pharmaceutically acceptable salt thereof;

(ii) memantine or a pharmaceutically acceptable salt thereof; and

(iii) instructions for simultaneous, separate or sequential use thereof for the treatment and/or prophylaxis of (a) a neurodegenerative disease; (b) dementia; (c) a mild cognitive impairment; (d) glaucoma; (e) ocular hypertension; or (f) pain, or for delaying the onset of (a) a neurodegenerative disease; (b) dementia; (c) a mild cognitive impairment; (d) glaucoma; (e) ocular hypertension; or (0 pain.

(7) The kit of (6), wherein (i) perampanel, a pharmaceutically acceptable salt thereof, a hydrate thereof, or a hydrate of a pharmaceutically acceptable salt thereof; and (ii) memantine or a pharmaceutically acceptable salt thereof are administered separately to the patient or are administered to the patient in the form of a pharmaceutical composition.

(8) Use of:

(i) perampanel, a pharmaceutically acceptable salt thereof, a hydrate thereof, or a hydrate of a pharmaceutically acceptable salt thereof; and

(ii) memantine or a pharmaceutically acceptable salt thereof for producing a pharmaceutical composition in the treatment of (a) a neurodegenerative disease; (b) dementia; (c) a mild cognitive impairment; (d) glaucoma; (e) ocular hypertension; or (f) pain.

(9) The use of (8), wherein (i) perampanel, a pharmaceutically acceptable salt thereof, a hydrate thereof, or a hydrate of a pharmaceutically acceptable salt thereof; and (ii) memantine or a pharmaceutically acceptable salt thereof are administered separately to the patient or are administered to the patient in the form of a pharmaceutical composition.

(10) A method for treating (a) a neurodegenerative disease; (b) dementia; (c) a mild cognitive impairment; (d) glaucoma; (e) ocular hypertension; or (f) pain, in a patient in need thereof comprising administering (i) perampanel, a pharmaceutically acceptable salt thereof, a hydrate thereof, or a hydrate of a pharmaceutically acceptable salt thereof; and (ii) memantine or a pharmaceutically acceptable salt thereof.

(11) The method of (10), wherein (i) perampanel, a pharmaceutically acceptable salt thereof, a hydrate thereof, or a hydrate of a pharmaceutically acceptable salt thereof; and (ii) memantine or a pharmaceutically acceptable salt thereof are administered separately to the patient or are administered to the patient in the form of a pharmaceutical composition.

(12) (i) A perampanel, a pharmaceutically acceptable salt thereof, a hydrate thereof, or a hydrate of a pharmaceutically acceptable salt thereof; and (ii) memantine or a pharmaceutically acceptable salt thereof in the treatment of (a) a neurodegenerative disease; (b) dementia; (c) a mild cognitive impairment; (d) glaucoma; (e) ocular hypertension; or (f) pain.

These and other aspects of the invention are described in more detail herein.

### BRIEF DESCRIPTION OF THE DRAWINGS

FIG. 1 shows the effects of perampanel (A) and memantin (B) on neuronal damage induced by combination of kainate and NMDA.

FIG. 2 shows the effects of perampanel, memantine alone and combination therapy on neuronal injury-induced by combination of kainate and NMDA.

### DETAILED DESCRIPTION OF THE INVENTION

“Patient” refers to animals, preferably mammals, more preferably humans. The term “patient” includes men and women; and includes adults, children and neonates. In one embodiment, the patient can be a companion animal, such as a dog or a cat.

“Active ingredient” refers to AMPA receptor antagonists and/or NMDA receptor antagonists.

“Treatment” and “treating” refer to the acquisition of a desired pharmacological effect and/or physiologic effect. These effects are prophylactic in terms of completely or partially preventing a disease and/or symptom(s), and therapeutic in terms of partially or completely curing a disease and/or an adverse event caused by a disease. “Treatment” and “treating” include any treatment of a disease in a patient including, for example: (a) to prevent a disease or symptom(s) in a patient who is suspected of being predisposed to the disease or symptom(s) but not yet diagnosed to be so; (b) to inhibit a symptom(s) of a disease, i.e., to inhibit or delay the progression of the symptom(s); and (c) to alleviate a symptom(s) of a disease, i.e., to reverse or eliminate the symptom(s) of the disease; or to reverse the progress of the symptom(s).

“Administered separately” with reference to the administration of two or more active ingredients to treat and/or prevent and/or delay the onset of the diseases described herein includes, for example, the sequential administration of the active ingredients in any order or the simultaneous administration of the active ingredients. Simultaneous administration of the active ingredients means that the active ingredients are administered to the patient at substantially the same time or at exactly the same time, depending on the mode of administration. The sequential administration of the active ingredients may occur in any order and may occur with any amount of time elapsing between administration of the active ingredients. Sequential administration may be based on factors that would influence which of the active ingredients should be administered first and which should be administered second, and how much time should elapse between administration of the active ingredients. For example, when two or more active ingredients are administered separately and sequentially, factors that effect when the active ingredients are administered to the patient include, for example, (a) the time(s) that provides the best efficacy for the active ingredient being administered, (b) the time(s) that provides the fewest side effects for the active ingredient being administered, (c) the dosage of the active ingredient, (d) the route of administration of the active ingredient, (e) the disease being treated, (f) the patient being treated, (g) the in vivo relationship of the active ingredients being administered, and other such factors known in the art. The time intervals for sequential administration are generally chosen so that the effect on the disease being treated in the combined use of the active ingredients is greater than additive when compared to the effect which would be obtained by use of only one of the active ingredients.

The term “combination” refers to the AMPA receptor antagonist and the NMDA receptor antagonist being administered separately as distinct pharmaceutical formulations (e.g., a first pharmaceutical formulation comprising an AMPA receptor antagonist and a second pharmaceutical formulation comprising an NMDA receptor antagonist). The pharmaceutical formulations can have the same or different modes of administration.

“Monotherapy” is a therapy which uses only one active ingredient for treatment and/or prophylaxis and/or delaying the onset of a disease.

“Combination therapy” is a therapy where two or more active ingredients are administered separately or are administered in the form of a pharmaceutical composition for the treatment and/or prophylaxis and/or delayed onset of a disease.

“Therapeutically effective amount” refers to the amount of the active ingredient that is necessary for the treatment and/or prophylaxis and/or delayed onset of a disease. When two or more active ingredients are administered as a pharmaceutical composition or for combination therapy, the term “therapeutically effective amount” refers to the amount of active ingredients that are necessary for treatment and/or prophylaxis and/or delayed onset of a disease and includes, for example: (a) a therapeutically effective amount of a first active ingredient and a therapeutically effective amount of a second active ingredient (i.e., the amount of each active ingredient that would be used for monotherapy for the treatment and/or prophylaxis of a disease is used for the pharmaceutical composition or combination therapy); (b) a therapeutically effective amount of a first active ingredient and a sub-therapeutic amount of a second active ingredient, which in combination effectively provide for treatment and/or prophylaxis of a disease (e.g., the sub-therapeutic amount of the second active ingredient can be used in a pharmaceutical composition or combination therapy to achieve a result that would be equal to or greater than the result that the second active ingredient would achieve if it was used for monotherapy); (b) a sub-therapeutic amount of a first active ingredient and a therapeutically effective amount of a second active ingredient, which in combination effectively provide for treatment and/or prophylaxis of a disease (e.g., the sub-therapeutic amount of the first active ingredient can be used in a pharmaceutical composition or combination therapy to achieve a result that would be equal to or greater than the result that the first active ingredient would achieve if it was used for monotherapy); and (d) a sub-therapeutic amount of a first active ingredient and a sub-therapeutic amount of a second active ingredient, which in combination therapy provide for treatment and/or prophylaxis of a disease or disorder (e.g., the sub-therapeutic amount of the first active ingredient can be used in a pharmaceutical composition or combination therapy to achieve a result that would be equal to or greater than the result that the first active ingredient would achieve if it was used for monotherapy; and the sub-therapeutic amount of the second active ingredient can be used in pharmaceutical composition or combination therapy to achieve a result that would be equal to or greater than the result that the second active ingredient would achieve if it was used for monotherapy).

“Kits” can include a combination of (i) a first pharmaceutical composition or formulation comprising the AMPA receptor antagonist; (ii) a second pharmaceutical composition or formulation comprising the NMDA receptor antagonist; (iii) instructions approved by the FDA for using the pharmaceutical compositions or formulations for treating or preventing or delaying the onset of the disease; and (iv) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The kit can supply enough medication and materials for days, weeks or months. In another embodiment, “kits” can include (i) pharmaceutical compositions or formulations comprising both the AMPA receptor antagonist and the NMDA receptor antagonist; (ii) instructions approved by the FDA for using the pharmaceutical compositions or formulations for treating or preventing or delaying the onset of the disease; and (iii) optionally other materials to administer the pharmaceutical compositions or formulations (e.g., syringes, diluents, medical gloves, hand sanitizers, and the like); to monitor drug levels in the body; to support patient compliance with medication dosing; or to monitor the status of the disease. The kit can supply enough medication and materials for days, weeks or months.

“Solvate” includes hydrate. The AMPA receptor antagonist may be in anhydride form, and may form a solvate such as a hydrate. The solvate may be either a hydrate or a nonhydrate, preferably a hydrate. The solvent used may be water, alcohol (e.g., methanol, ethanol or n-propanol), dimethylformamide or the like.

“Hydrate” refers to an active ingredient or compound containing a molecule of water of crystallization. The molecule of water of crystallization can be an integer of 1 or more, such as 1 to 10; or can be any fraction greater than 0 or a fraction of an integer from 1 to 10. For example, the hydrate may be represented as (active ingredient)·¼H₂O; (active ingredient)·½H₂O; (active ingredient)·¾H₂O; (active ingredient)·2H₂O; (active ingredient)·5½H₂O; (active ingredient)·6H₂O; and the like.

“Pharmaceutically acceptable salts” are well known in the art and include those of inorganic acids, such as hydrochloride, sulfate, hydrobromide and phosphate; and those of organic acids, such as formate, acetate, trifluoroacetate, methanesulfonate, benzenesulfonate and toluenesulfonate. When certain substituents are selected, the active ingredients can form, for example, alkali metal salts, such as sodium or potassium salts; alkaline earth metal salts, such as calcium or magnesium salts; organic amine salts, such as a salt with trimethyl-amine, triethylamine, pyridine, picoline, dicyclohexylamine or N,N′-dibenzylethylenediamine. One skilled in the art will recognize that the active ingredients can be made in the form of any other pharmaceutically acceptable salt.

“Dementia” refers to a deterioration of intellectual functioning, and is characterized by one or more symptoms of cognitive impairments, disorientation, impaired memory, impaired judgment, impaired intellect, and the like. “Dementia” may also include behavioral disturbances. Exemplary behavioral disturbances include sexual disinhibition, changes in activity, changes in interpersonal relationships, physical aggressiveness, physical non-aggressiveness (e.g., wandering), verbal aggressiveness, and verbal non-aggressiveness (e.g., repetitive vocalization). With respect to dementia, the methods of the invention may be used to treat, prevent, or delay the onset of (i) intellectual functioning associated with dementia; (ii) behavioral disturbances associated with dementia; or (iii) intellectual functioning and behavioral disturbances associated with dementia. The cause(s) of dementia may be known or unknown. For example, dementia may be caused by a neurodegenerative disease, Alzheimer's disease, Parkinson's disease, Huntington's disease, Pick's disease, Lewy body disease, vascular disease (e.g., cerebrovascular disease), HIV/AIDS, epilepsy, brain tumors, brain lesions, multiple sclerosis, Down's syndrome, Rett's syndrome, progressive supranuclear palsy, frontal lobe syndrome, schizophrenia, traumatic brain injuries (e.g., closed head injuries), post coronary artery by-pass graft surgery, electroconvulsive shock therapy, chemotherapy, radiation therapy, radiation exposure, encephalitis, meningitis, fetal alcohol syndrome, Korsakoffs syndrome, anoxic brain injury, cardiopulmonary resuscitation, diabetes, menopause, strokes, high cholesterol levels, or spinal cord disorders. For additional descriptions of dementia, the disclosures of U.S. Pat. No. 6,458,807, US Publication No. 2006/0018839, and WO 2005/074535 are incorporated by reference herein in their entirety.

“Mild cognitive impairments” are a transition stage between the cognitive changes of normal aging and the more serious problems caused by Neurodegenerative disease. While mild cognitive impairments can affect many areas of cognition—language, attention, reasoning, judgment, reading and writing—most research has focused on its effects on memory. The disorder can be divided into two broad subtypes. Amnestic mild cognitive impairments significantly affect memory, while nonamnestic mild cognitive impairments do not. Other functions, such as language and attention span, may be impaired in either subtype.

“Neurodegenerative disease” refers to any neurodegenerative disease known in the art. Exemplary neurodegenerative diseases include Alzheimer's disease, Parkinson's disease, Huntington's disease, amyotrophic lateral sclerosis, multiple sclerosis, Pick's disease, Lewy body disease, prion diseases (e.g., Creutzfeldt-Jakob disease), epilepsy, strokes, and the like.

“Alzheimer's disease” refers to and includes one or more of mild Alzheimer's disease; mild-to-moderate Alzheimer's disease; moderate Alzheimer's disease; moderate-to-severe Alzheimer's disease; and severe Alzheimer's disease. Clinical symptoms of Alzheimer's disease include progressive disorientation, amnesia, and aphasia, which eventually cause incompetence, speech loss, and akinesia. Examples of pathological signs of Alzheimer's disease include neurofibrillary tangle, senile plaque, and amyloid vascular disorder. “To prevent progression of Alzheimer's disease” refers to preventing the onset or further progression of a clinical symptom(s) and/or a pathological sign(s) of Alzheimer's disease. For example, progression of a clinical symptom or a pathological sign can be prevented for patients who do not exhibit the clinical symptom(s) or pathological sign(s) of Alzheimer's disease. In addition, patients with a milder form of Alzheimer's disease can be prevented from progressing to a more severe form of Alzheimer's disease. “To delay the progression of Alzheimer's disease” refers to delaying the onset of a symptom(s) and/or pathological sign(s) of Alzheimer's disease; or to slow down the rate at which Alzheimer's disease progresses, as determined by a clinical symptom(s) and/or a pathological sign(s). “To reverse the progression of Alzheimer's disease” refers to alleviating the severity of a symptom(s) of Alzheimer's disease, i.e., to alter the patient's symptom(s) from a more severe to a more mild condition, as determined by a reduction of the clinical symptom(s) and/or the pathological sign(s).

An Alzheimer's disease diagnosis can be carried out using various known methods. Typically, clinical and pathological assessments are combined to diagnose a patient with Alzheimer's disease. For example, progression or severity of Alzheimer's disease can be assessed using the Mini Mental State Examination (MMSE)(Mohs et al, (1996) Int. Psychogeriatr 8:195-203); the Alzheimer's Disease Assessment Scale-Cognitive Subscale (ADAS-cog)(Galasko et al, (1997) Alzheimer Dis. Assoc. Disord., 11 Suppl. 2:S33-39); the Alzheimer's Disease Cooperative Study-Activities of Daily Living scale (ADCS-ADL) (McKhann et al, (1984) Neurology 34:939-944); and the National Institute of Neurologic Communicative Disorders and the Stroke-Alzheimer's Disease and Related Disorders Association (NINCDS-ADRDA) criteria (Folstein et al, (1975) J. Psychiatr. Res. 12:189-198; McKhann et al, (1984) Neurology 34:939-944). Additionally, methods capable of assessing various areas in the brain to estimate frequency of senile plaque or neurofibrillary tangle may be used (Braak et al (1991) Acta Neuropathol 82:239-259; Khachaturian (1985) Arch Neuro 42:1097-1105; Mirra et al, (1991) Neurology, 41:479-486; Mirra et al, (1993) Arch Pathol Lab Med 117:132-144).

“Ocular hypertension” sometimes referred to as “glaucoma suspect” refers to any situation in which the pressure inside the eye, called intraocular pressure, is higher than normal. Eye pressure is measured in millimeters of mercury (mm Hg). Normal eye pressure ranges from 10-21 mm Hg. Ocular hypertension is an eye pressure greater than 21 mm Hg. Ocular hypertension is generally not associated with optic nerve damage or vision loss. Ocular hypertension may in some circumstances lead to glaucoma.

“Glaucoma” is associated with symptoms of optic nerve damage and vision loss. Glaucoma is generally, but not always, accompanied by increased intraocular pressure. Types of glaucoma include primary open angle glaucoma, angle closure glaucoma, normal tension glaucoma, and pediatric glaucoma.

“Pain” refers to any type of pain known in the art. In one embodiment, “pain” refers to neuropathic pain. In another embodiment, “pain” refers to headaches.

“Neuropathic pain” refers to a complex, chronic pain state that usually is accompanied by tissue injury. With neuropathic pain, the nerve fibers themselves may be damaged, dysfunctional or injured. These damaged nerve fibers send incorrect signals to other pain centers. The impact of nerve fiber injury includes a change in nerve function both at the site of injury and areas around the injury. Symptoms of neuropathic pain may include shooting and burning pain and/or tingling and numbness. One example of neuropathic pain is called phantom limb syndrome. This occurs when an arm or a leg has been removed because of illness or injury, but the brain still gets pain messages from the nerves that originally carried impulses from the missing limb. These nerves now misfire and cause pain. Neuropathic pain often seems to have no obvious cause; but, some exemplary causes of neuropathic pain include: alcoholism; amputation; back, leg, and hip problems; chemotherapy; diabetes; facial nerve problems; HIV infection; AIDS; multiple sclerosis; shingles; spine surgery, and the like.

“Headaches” refer to primary headaches and secondary headaches according to The International Classification of Headache Disorders as described in Cephalagia, 24(Suppl. 1):9-160 (2004), the disclosure of which is incorporated by reference herein in its entirety. The primary headaches and secondary headaches may be episodic or chronic. “Episodic headaches” refers to patients who experience headaches from 1 to 14 days per month. “Chronic headaches” refers to patients who experience headaches 15 or more days per month. The methods of treatment may be acute or chronic. “Acute treatment” refers to treating a headache on an as-need basis, e.g., upon the onset of the headache a patient is administered a therapeutically effective amount of the compounds or compositions of the invention as described herein. “Chronic treatment” refers to treating a headache on a continual (e.g., daily) basis whether or not the patient is experiencing a headache at the time of administration of the therapeutically effective amount of the compounds or compositions of the invention as described herein. Headaches may cause one or more symptoms such as vertigo, nausea, vomiting, fatigue, aura, photophobia, phonophobia, and the like.

“Primary headaches” include migraines, tension headaches, cluster headaches, paroxysmal hemicrania, short-lasting unilateral neuralgiform headache attacks with conjunctival injection and tearing (SUNCT), trigeminal autonomic cephalalgia, stabbing headaches, cough headaches, exertional headaches, headaches associated with sexual activity, hypnic headaches, thunderclap headaches, hemicrania continua, or a new daily-persistent headache.

“Secondary headaches” include headaches attributed to head and/or neck traumas; headaches attributed to cranial and/or cervical vascular disorders; headaches attributed to non-vascular intracranial disorders; headaches attributed to drugs; headaches attributed to withdrawal from drugs; headaches attributed to infections, headaches attributed to disturbances of homeostasis, headaches or facial pain attributed to disorders of facial structures and/or cranial structures; or headaches attributed to a psychiatric disorders.

“Migraine” refers to a symptom complex occurring periodically and characterized by pain in the head, usually unilateral pain in the head. Migraines generally have a pulsating quality and moderate or severe intensity that inhibits or prohibits daily activities. One or more symptoms caused by migraines include vertigo, nausea, vomiting, fatigue, aura, photophobia, phonophobia, and the like. Migraines may occur with or without aura. Exemplary migraines include classic migraines, common migraines, complicated migraines, menstrual migraines, premenstrual migraines, ophthalmic migraines, opthalmoplegic migraines, fulgurating migraines, Harris' migraines, and hemiplegic migraines. Neurologic symptoms can occur which are caused by migraines, but which are not followed by head pain. For example, abdominal pain and vomiting can occur without head pain as the sole expression of a migraine.

The invention provides pharmaceutical compositions comprising a therapeutically effective amount of: (i) at least one AMPA receptor antagonist; (ii) at least one NMDA receptor antagonist; and (iii) one or more pharmaceutically acceptable carriers.

In other embodiments, the invention provides combinations comprising a therapeutically effective amount of: (i) at least one AMPA receptor antagonist and (ii) at least one NMDA receptor antagonist. The combination may be administered separately (e.g., simultaneously, sequentially) to a patient to treat the diseases described herein.

In other embodiments, the invention provides kits comprising a therapeutically effective amount of: (i) at least one AMPA receptor antagonist and (ii) at least NMDA receptor antagonist; and (iii) instructions for simultaneous, separate or sequential use thereof in the treatment of the diseases described herein.

In other embodiments, the invention provides kits comprising a therapeutically effective amount of pharmaceutical compositions comprising at least one AMPA receptor antagonist and at least NMDA receptor antagonist; and instructions for the use thereof in the treatment of the diseases described herein.

In other embodiments, the invention provides (i) methods for treating or preventing a neurodegenerative disease; and (ii) methods for delaying the onset of a neurodegenerative disease in a patient in need thereof by administering a therapeutically effective amount of: (a) at least one AMPA receptor antagonist and (b) at least NMDA receptor antagonist. The AMPA receptor antagonist and the NMDA receptor antagonist can be administered separately or can be administered in the form of a composition. The neurodegenerative disease may be any known in the art, such as those described herein.

In other embodiments, the invention provides (i) methods for treating or preventing Alzheimer's disease; and (ii) methods for delaying the onset of Alzheimer's disease in a patient in need thereof by administering a therapeutically effective amount of: (a) at least one AMPA receptor antagonist and (b) at least one NMDA receptor antagonist. The AMPA receptor antagonist and the NMDA receptor antagonist can be administered separately or can be administered in the form of a composition.

The invention provides (i) methods for treating or preventing dementia in a patient in need thereof; and (ii) methods for delaying the onset of dementia in a patient in need thereof by administering a therapeutically effective amount of: (a) at least one AMPA receptor antagonist and (b) at least one NMDA receptor antagonist. The AMPA receptor antagonist and the NMDA receptor antagonist can be administered separately or can be administered in the form of a composition.

The invention provides (i) methods for treating or preventing mild cognitive impairments in a patient in need thereof; and (ii) methods for delaying the onset of mild cognitive impairments in a patient in need thereof by administering a therapeutically effective amount of: (a) at least one AMPA receptor antagonist and (b) at least one NMDA receptor antagonist. The AMPA receptor antagonist and the NMDA receptor antagonist can be administered separately or can be administered in the form of a composition.

The invention provides methods for the treatment and/or prophylaxis of glaucoma or ocular hypertension in a patient in need thereof by administering (i) at least one AMPA receptor antagonist and (ii) at least one NMDA receptor antagonist. The invention provides methods for delaying the onset of glaucoma or ocular hypertension by administering (i) at least one AMPA receptor antagonist and (ii) at least one NMDA receptor antagonist. In one embodiment, the active ingredients may be topically administered to the affected eye in the form of a gel or liquid (e.g., solution, suspension, and the like). The methods generally involve lowering intraocular pressure in the eye(s) of a patient in need thereof.

The invention provides methods for the treatment and/or prophylaxis of pain in a patient in need thereof by administering (i) at least one AMPA receptor antagonist and (ii) at least one NMDA receptor antagonist. The invention provides methods for delaying the onset of pain by administering (i) at least one AMPA receptor antagonist and (ii) at least one NMDA receptor antagonist. In various embodiments, the pain may be neuropathic pain, headaches, and the like.

The invention provides methods of treating, preventing, or delaying the onset of canine cognitive dysfunctions (CCD), which are the age related deterioration of cognitive abilities in dogs characterized by behavioral changes that cannot be wholly attributed to general medical conditions. When preparing veterinary compositions, one skilled in the art would appreciate that the composition would contain a therapeutically effective amount of: (a) at least one AMPA receptor antagonist, (b) at least one NMDA receptor antagonist, and (c) a veterinarily acceptable carrier. The dosage amount for a dog can be easily determined based on the dosages that are acceptable for humans, the size and weight of the dog, the dogs medical condition, and the like.

In one embodiment, the AMPA receptor antagonist used in the methods and compositions described herein may be any known in the art. Exemplary AMPA receptor antagonists, all of which are active ingredients, include 1,2-dihydropyridine compounds, quinoxalinedione aminoalkylphosphonates, and the like.

In one embodiment, the AMPA receptor antagonist is a 1,2-dihydropyridine compound. The 1,2-dihydropyridine compound used in the methods and compositions described herein may be any known in the art. “1,2-dihydropyridine compound” includes 1,2-dihydropyridine compounds, pharmaceutically acceptable salts of 1,2-dihydropyridine compounds, stereoisomers of 1,2-dihydropyridine compounds, pharmaceutically acceptable salts of stereoisomers of 1,2-dihydropyridine compounds, hydrates of 1,2-dihydropyridine compounds, hydrates of pharmaceutically acceptable salts of 1,2-dihydropyridine compounds, stereoisomers of hydrates of 1,2-dihydropyridine compounds, and stereoisomer of hydrates of pharmaceutically acceptable salts of 1,2-dihydropyridine compounds.

The 1,2-dihydropyridine compound used in the methods and compositions described herein may be a compound of Formula (I):

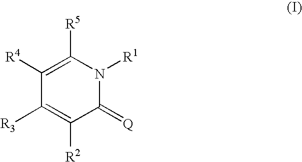

wherein Q is NH, O or S; R¹, R², R³, R⁴ and R⁵ are each independently hydrogen, halogen, C₁₋₆ alkyl, or —X-A; X is a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ⁿ—N(R¹²)—, —CH₂—S(O)ₚ—, —S(O)_{q}—CH₂—, —CH₂—O—, —O—CH₂—, —N(R¹³)—CO—N(R¹⁴)— or —N(R¹⁵)—CS—N(R¹⁶)—, R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A is an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring; provided that 3 groups among R¹, R², R³, R⁴ and R⁵ are —X-A; and that the residual 2 groups among R¹, R², R³, R⁴ and R⁵ are independently hydrogen, halogen, or C₁₋₆ alkyl.

In one embodiment, the following compounds are excluded from the scope of the compound of Formula (I): (1) when Q is O; R¹ and R⁵ are hydrogen; and R², R³ and R⁴ are phenyl; (2) when Q is O; R¹ and R⁴ are hydrogen; and R², R³ and R⁵ are phenyl; and (3) when Q is O; R¹ and R² are hydrogen; and R³, R⁴ and R⁵ are phenyl.

The 1,2-dihydropyridine compound used in the methods and compositions described herein may be a compound of Formula (II):

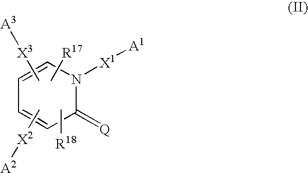

wherein Q is NH, O or S; X¹, X² and X³ are each independently a single bond, an optionally substituted C₁₋₆ alkylene, an optionally substituted C₂₋₆ alkenylene, an optionally substituted C₂₋₆ alkynylene, —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)_{P}, —S(O)_{q}—CH₂—, —CH₂—O—, —O—CH₂—, —N(R¹³)—CO—N(R¹⁴)— or —N(R¹⁵)—CS—N(R¹⁶); R⁶, R⁷, R⁸, R⁹, R¹⁰, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5 to 14-membered aromatic heterocyclic ring; and R¹⁷ and R¹⁸ are each independently hydrogen, halogen, or C₁₋₆ alkyl.

The invention provides the compound of Formula (II) wherein X¹, X² and X³ are each independently a single bond, an optionally substituted C₁₋₆ alkylene, an optionally substituted C₂₋₆ alkenylene, or an optionally substituted C₂₋₆ alkynylene. The substituents may be one or more of —O—, —S—, —CO—, —SO—, —SO₂—, —N(R⁶)—, —N(R⁷)—CO—, —CO—N(R⁸)—, —N(R⁹)—CH₂—, —CH₂—N(R¹⁰)—, —CH₂—CO—, —CO—CH₂—, —N(R¹¹)—S(O)ₘ—, —S(O)ₙ—N(R¹²)—, —CH₂—S(O)_{P}—, —S(O)_{q}—CH₂—, —CH₂—O—, —O—CH₂—, —N(R¹³)—CO—N(R¹⁴)— and —N(R¹⁵)—CS—N(R¹⁶)—; R⁶, R⁷, R⁸, R⁹, R¹⁰, R¹¹, R¹², R¹³, R¹⁴, R¹⁵ and R¹⁶ are each independently hydrogen, C₁₋₆ alkyl, or C₁₋₆ alkoxy; m, n, p and q are each independently an integer of 0, 1 or 2; A¹, A² and A³ are each independently an optionally substituted C₃₋₈ cycloalkyl, an optionally substituted C₃₋₈ cycloalkenyl, an optionally substituted 5- to 14-membered non-aromatic heterocyclic ring, an optionally substituted C₆₋₁₄ aromatic hydrocarbocyclic ring, or an optionally substituted 5- to 14-membered aromatic heterocyclic ring.

The substituents for the 1,2-dihydropyridine compounds of the invention may be one or more of hydroxy; halogen; nitrile; nitro; C₁₋₆ alkyl; C₂₋₆ alkenyl; C₂₋₆ alkynyl [wherein the alkyl, alkenyl, and alkynyl can independently and optionally be substituted with one or more groups selected from hydroxy, nitrile, halogen, C₁₋₆ alkylamino, di(C₁₋₆ alkyl) amino, C₂₋₆ alkenylamino, di(C₂₋₆ alkenyl)amino, C₂₋₆ alkynylamino, di(C₂₋₆ alkynyl)amino, N—C₁₋₆ alkyl-N—C₂₋₆ alkenylamino, N—C₁₋₆ alkyl-N—C₂₋₆ alkynylamino, N—C₂₋₆ alkenyl-N—C₂₋₆alkynylamino, aralkyloxy, TBDMS oxy, C₁₋₆ alkylsulfonylamino, C₁₋₆ alkylcarbonyloxy, C₂₋₆ alkenylcarbonyloxy, C₂₋₆ alkynylcarbonyloxy, N—C₁₋₆ alkylcarbamoyl, N—C₂₋₆ alkenylcarbamoyl, and N—C₁₋₆ alkynylcarbamoyl]; C₁₋₆ alkoxy; C₂₋₆ alkenyloxy; C₂₋₆ alkynyloxy [wherein the alkoxy, alkenyloxy, and alkynyloxy may independently and optionally be substituted with one or more groups selected from C₁₋₆ alkylamino, aralkyloxy, and hydroxy]; C₁₋₆ alkylthio; C₂₋₆ alkenylthio; C₂₋₆ alkynylthio [wherein the alkylthio, alkenylthio, and alkynylthio may independently and optionally be substituted with one or more groups selected from hydroxy, nitrile, halogen, C₁₋₆ alkylamino, aralkyloxy, TBDMS oxy, C₁₋₆ alkylsulfonylamino, C₁₋₆ alkylcarbonyloxy, and C₁₋₆ alkylcarbamoyl]; optionally substituted carbonyl [which may be substituted with C₁₋₆ alkoxy, amino, C₁₋₆ alkylamino, alkyl)amino, C₂₋₆ alkenylamino, di(C₂₋₆ alkenyl)amino, C₂₋₆ alkynylamino, di(C₂₋₆ alkynyl)amino, N—C₁₋₆ alkyl-N—C₂₋₆ alkenylamino, N—C₁₋₆ alkyl-N—C₂₋₆ alkynylamino and N—C₂₋₆ alkenyl-N—C₂₋₆ alkynylamino]; an optionally substituted amino [which may be substituted with one or two groups selected from C₁₋₆ alkyl, C₂₋₆ alkenyl, C₂₋₆ alkynyl, C₁₋₆ alkylsulfonyl, C₂₋₆ alkenylsulfonyl, C₂₋₆ alkynylsulfonyl, C₁₋₆ alkylcarbonyl, C₂₋₆ alkenylcarbonyl and C₂₋₆ alkynylcarbonyl]; C₁₋₆ alkylsulfonyl; C₂₋₆ alkenylsulfonyl; C₂₋₆ alkynylsulfonyl; C₁₋₆alkylsulfinyl; C₂₋₆ alkenylsulfinyl; C₂₋₆ alkynylsulfinyl; formyl; optionally substituted C₃₋₈ cycloalkyl; an optionally substituted C₃₋₈ cycloalkenyl [where the cycloalkyl group and/or the cycloalkenyl group may independently and optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; a 5- to 14-membered non-aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; C₆₋₁₄ aromatic hydrocarbocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl]; and a 5- to 14-membered aromatic heterocyclic ring [which may optionally be substituted with one or more groups selected from hydroxy, halogen, nitrile, C₁₋₆ alkyl, C₁₋₆ alkyloxy, C₁₋₆ alkyloxy C₁₋₆ alkyl, and aralkyl].

In another embodiment, the invention provides compounds of Formula (II) wherein A¹, A² and A³ are each independently an optionally substituted C₃₋₈ cycloalkyl, an optionally substituted C₃₋₈ cycloalkenyl or an optionally substituted 5- to 14-membered non-aromatic hetero ring. In another embodiment, the invention provides the compound of Formula (II) wherein A¹, A² and A³ are each independently an optionally substituted C₆₋₁₄ aromatic hydrocarbon ring or an optionally substituted 5- to 14-membered aromatic hetero ring. In another embodiment, the invention provides the compound of Formula (II) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl or morpholyl; any of which may optionally have substituents. In another embodiment, the invention provides the compound of Formula (II) wherein A¹, A² and A³ are each independently selected from:

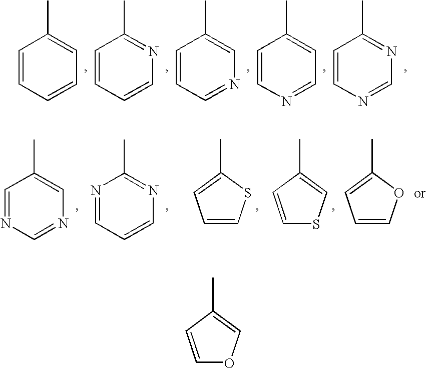

each of which may optionally be substituted. In another embodiment, the invention provides the compound of Formula (II) wherein A¹, A² and A³ are each independently substituted with hydroxyl, halogen, amino, or nitrile. In another embodiment, the invention provides the compound of Formula (II) wherein A¹, A² and A³ are each independently hydroxyl, halogen, amino, nitrile, or nitro. In another embodiment, the invention provides the compound of Formula (II) wherein Q is oxygen.

In another embodiment, the invention provides the compounds of Formula (I) or (II) wherein X', X² and X³ are each independently a single bond, —CH₂—, —CH(OH)—, —CH₂—CH₂—, —CH═CH—, —O— or —CO—. In another embodiment, the invention provides the compounds of Formula (I) or (II) wherein X¹, X² and X³ are each a single bond. In another embodiment, the invention provides the compounds of Formula (I) or (II) wherein R¹⁷ and R¹⁸ are each independently hydrogen, fluorine, chlorine, bromine, iodine, methyl, ethyl, n-propyl, or iso-propyl. In another embodiment, the invention provides the compounds of Formula (I) or (II) wherein R¹⁷ and R¹⁸ are each hydrogen.

“Halogen atom” indicates fluorine, chlorine, bromine, iodine and the like, and the preferable halogen atoms include fluorine, chlorine and bromine.

“C₁₋₆ alkyl” refers to an alkyl group having 1 to 6 carbons, and examples include linear chain or branched chain alkyl groups such as methyl, ethyl, n-propyl, iso-propyl, n-butyl, iso-butyl, sec-butyl (1-methylpropyl), tert-butyl, iso-pentyl, n-pentyl, tert-pentyl (1,1-dimethylpropyl), 1,2-dimethylpropyl, 2,2-dimethylpropyl (neopentyl), 1-ethylpropyl, 2-methylbutyl, n-hexyl, iso-hexyl, 1,2-dimethylbutyl, 1-ethyl-1-methylpropyl, 1-ethyl-2-methylpropyl, 1,1,2-trimethylpropyl, 1,2,2-trimethylpropyl, 1-ethylbutyl, 1-methylbutyl, 1,1-dimethylbutyl, 2,2-dimethylbutyl, 1,3-dimethylbutyl, 2,3-dimethylbutyl, 3,3-dimethylbutyl, 2-ethylbutyl, 1-methylpentyl, 2-methylpentyl, 3-methylpentyl, and the like.

“C₂₋₆ alkenyl” refers to an alkenyl group having 2 to 6 carbons, and examples include vinyl, 1-ethylethenyl (1-buten-2-yl), allyl (2-propenyl), 1-propenyl, iso-propenyl, 2-methyl-1-propenyl, 1-methyl-1-propenyl (2-buten-2-yl), 2-methyl-2-propenyl, 1-methyl-2-propenyl, 1-butenyl (1-buten-1-yl), 2-butenyl (2-buten-1-yl), 3-butenyl, 1-pentenyl, 1-hexenyl, 1,3-hexadienyl, 1,6-hexadienyl, and the like.

“C₂₋₆ alkynyl” refers to an alkynyl group having 2 to 6 carbons, and examples include ethynyl, 1-propynyl, 2-propynyl, 1-butynyl, 2-butynyl, 3-butynyl, 1-ethyl-1-propynyl, 1-ethynyl-2-propynyl, 2-methyl-3-butenyl, 1-pentynyl, 1-hexynyl, 1,3-hexadiynyl, 1,6-hexadiynyl, and the like.

“C₁₋₆ alkoxy” refers to an alkoxy group having 1 to 6 carbons, and examples include methoxy, ethoxy, n-propoxy, iso-propoxy, n-butoxy, iso-butoxy, sec-butoxy, tert-butoxy, n-pentyloxy, iso-pentyloxy, tert-pentyloxy, 1,2-dimethylpropoxy, neopentyloxy, 1-ethylpropoxy, 1-methylbutoxy, 2-methylbutyoxy, n-hexyloxy, iso-hexyloxy, 1-ethyl-1-methylpropoxy, 1-ethyl-2-methylpropoxy, 1,1,2-trimethylpropoxy, 1,2,2-trimethylpropoxy, 1,1-dimethylbutoxy, 2,2-dimethylbutoxy, 2,3-dimethylbutoxy, 3,3-dimethylbutoxy, 2-ethylbutoxy, 1,3-dimethylbutoxy, 1-ethylbutoxy, 1-methylbutoxy, 1-methylpentyloxy, 2-methylpentyloxy, 3-methylpentyloxy, and the like.

“C₂₋₆ alkynyloxy” refers to an alkynyloxy group having 2 to 6 carbon atoms, and examples include ethynyloxy, 1-propynyloxy, 2-propynyloxy, 1-butynyloxy, 2-butynyloxy, 3-butynyloxy, 1-methyl-2-propynyloxy, 1-ethyl-2-propynyloxy, 1-ethynyl-2-propynyloxy, 1-pentynyloxy, 1-hexynyloxy, 1,3-hexadiynyloxy, 1,6-hexadiynyloxy, and the like.

“C₂₋₆ alkenyloxy” refers to an alkenyloxy group having 2 to 6 carbons, and examples include vinyloxy, 1-ethylethenyloxy (1-buten-2-yloxy), allyloxy (2-propenyloxy), 1-propenyloxy, iso-propenyloxy, 2-methyl-1-propenyloxy, 1-methyl-1-propenyloxy (2-buten-2-yloxy), 2-methyl-2-propenyloxy, 1-methyl-2-propenyloxy (1-buten-3-yloxy), 1-butenyloxy (1-buten-1-yloxy), 2-butenyloxy (2-buten-1-yloxy), 3-butenyloxy, 1-pentenyloxy, 1-hexenyloxy, 1,3-hexadienyloxy, 1,6-hexadienyloxy, and the like.

“C₃₋₈ cycloalkyl” refers to a cycloalkyl group composed of 3 to 8 carbon atoms, and examples include cyclopropyl, cyclobutyl, cyclopentyl, cyclohexyl, cycloheptyl, cyclooctyl, and the like.

“C₃₋₈ cycloalkenyl” refers to a cycloalkenyl group composed of 3 to 8 carbon atoms, and examples include cyclopropen-1-yl, 2-cyclopropen-1-yl, cyclobuten-1-yl, 2-cyclobuten-1-yl, 1,3-cyclobutadien-1-yl, cyclopenten-1-yl, 2-cyclopenten-1-yl, 3-cyclopenten-1-yl, 1,3-cyclopentadien-1-yl, 1,4-cyclopentadien-1-yl, 2,4-cyclopentadien-1-yl, cyclohexen-1-yl, 2-cyclohexen-1-yl, 3-cyclohexen-1-yl, 1,3-cyclohexadien-1-yl, 1,4-cyclohexadien-1-yl, 1,5-cyclohexadien-1-yl, 2,4-cyclohexadien-1-yl, 2,5-cyclohexadien-1-yl, cyclohepten-1-yl, 2-cyclohepten-1-yl, 3-cyclohepten-1-yl, 4-cyclohepten-1-yl, 1,3-cyclopentadien-1-yl, 1,4-cyclopentadien-1-yl, 1,5-cycloheptadien-1-yl, 1,6-cycloheptadien-1-yl, 2,4-cycloheptadien-1-yl, 2,5-cycloheptadien-1-yl, 2,6-cycloheptadien-1-yl, 3,5-cycloheptadien-1-yl, 1,3,5-cycloheptatrien-1-yl, 1,3,6-cycloheptatrien-1-yl, 1,4,6-cycloheptatrien-1-yl, 2,4,6-cycloheptatrien-1-yl, cycloocten-1-yl, 2-cycloocten-1-yl, 3-cycloocten-1-yl, 4-cycloocten-1-yl, 1,3-cyclooctadien-1-yl, 1,4-cyclooctadien-1-yl, 1,5-cyclooctadien-1-yl, 1,6-cyclooctadien-1-yl, 1,7-cyclooctadien-1-yl, 2,4-cyclooctadien-1-yl, 2,5-cyclooctadien-1-yl, 2,6-cyclooctadien-1-yl, 2,7-cyclooctadien-1-yl, 3,5-cyclooctadien-1-yl, 3,6-cyclooctadien-1-yl, 1,3,5-cyclooctatrien-1-yl, 1,3,6-cyclooctatrien-1-yl, 1,3,7-cyclooctatrien-1-yl, 1,4,6-cyclooctatrien-1-yl, 1,4,7-cyclooctatrien-1-yl, 1,5,7-cyclooctatrien-1-yl, 2,4,6-cyclooctatrien-1-yl, 2,4,7-cyclooctatrien-1-yl group, and the like.

“5- to 14-membered non-aromatic heterocyclic ring” refers to a mono-cyclic, di-cyclic, or tri-cyclic 5- to 14-membered non-aromatic heterocyclic ring which contains one or more hetero atoms selected from nitrogen, sulfur, and oxygen. Specific examples include pyrrolidinyl, pyrrolinyl, piperidyl, piperazinyl, pyrazolidinyl, imidazolidinyl, morpholinyl, tetrahydrofuryl, tetrahydropyranyl, dihydrofuryl, dihydropyranyl, imidazolinyl, oxazolinyl, and the like. Further, a group derived from a pyridone ring and a non-aromatic condensed ring (for example, a group derived from a phthalimide ring, a succinimide ring, and the like) are also included in the non-aromatic heterocyclic ring.

“C₆₋₁₄ aromatic hydrocarbocyclic ring and the aryl” refers to an aromatic hydrocarbocyclic ring which is composed of 6 to 14 carbon atoms, a mono-cyclic ring, and a condensed di-cyclic, tri-cyclic and the like. Specific examples include phenyl, indenyl, 1-naphthyl, 2-naphthyl, azulenyl, heptalenyl, biphenyl, indathenyl, acenaphthyl, fluorenyl, phenalenyl, phenanthrenyl, anthracenyl, cyclopentacyclooctenyl, benzocyclooctenyl and the like.

“5- to 14-membered aromatic heterocyclic ring and the heteroaryl ring” refers to mono-cyclic, di-cyclic, or tri-cyclic 5- to 14-membered aromatic heterocyclic ring which contain one or more hetero atoms selected from nitrogen, sulfur, and oxygen. Specific examples include (1) aromatic heterocyclic rings containing nitrogen such as pyrrolyl, pyridyl, pyridazinyl, pyrimidinyl, pyrazinyl, triazolyl, tetrazolyl, benzotriazolyl, pyrazolyl, imidazolyl, benzimidazolyl, indolyl, iso-indolyl, indolizinyl, prenyl, indazolyl, quinolyl, iso-quinolyl, quinoliziyl, phthalazyl, naphthylidinyl, quinoxalyl, quinazolinyl, cynnolinyl, pteridinyl, imidazotriazinyl, pyrazinopyridazinyl, acridinyl, phenanthridinyl, carbazolyl, carbazolinyl, perimidinyl, phenanthrolinyl, phenazinyl, imidazopyridyl, imidazopyrimidinyl, or pyrazolopyridyl; (2) aromatic heterocyclic rings containing sulfur such as thienyl or benzothienyl; (3) aromatic heterocyclic rings containing oxygen such as furyl, pyranyl, cyclopentapyranyl, benzofuryl, or iso-benzofuryl; and (4) aromatic heterocyclic rings containing 2 or more different hetero atoms such as thiazolyl, iso-thiazolyl, benzothiazolyl, benzothiadiazolyl, phenothiazinyl, isoxazolyl, furazanyl, phenoxazinyl, oxazolyl, isoxazoyl, benzoxazolyl, oxadiazolyl, pyrazoloxadiazolyl, imidazothiazolyl, thienofuranyl, furopyrrolyl or pyridoxadinyl.

The 1,2-dihydropyridine compound used in the methods and compositions described herein may be a compound of Formula (III):

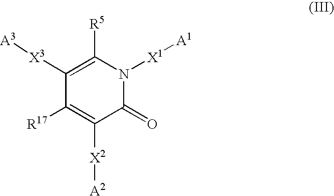

wherein X¹, X², X³, A¹, A², A³, R¹⁷ and R¹⁸ have the same meanings as defined in the above compound of Formula (II).

In another embodiment, the invention provides the compounds of Formula (III) wherein A¹, A² and A³ are each independently an optionally substituted C₆₋₁₄ aromatic hydrocarbon ring or 5- to 14-membered aromatic hetero ring. In another embodiment, the invention provides the compounds of Formula (III) wherein A¹, A² and A³ are each independently phenyl, pyrrolyl, pyridyl, pyridazinyl, pyrimidinyl, pyrazinyl, thienyl, thiazolyl, furyl, naphthyl, quinolyl, iso-quinolyl, indolyl, benzimidazolyl, benzothiazolyl, benzoxazolyl, imidazopyridyl, carbazolyl, cyclopentyl, cyclohexyl, cyclohexenyl, dioxinyl, adamantyl, pyrrolidinyl, piperidinyl, piperazinyl, or morpholyl; wherein each may optionally be substituted. In another embodiment, the invention provides the compounds of Formula (III) wherein A¹, A² and A³ are each independently selected from:

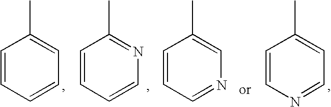

each of which may optionally be substituted. In another embodiment, the invention provides the compounds of Formula (III) wherein the bonding site of the substituent at A¹, A² and A³ are in the α-position of the carbon atom bonding to the group X¹, X² and X³, respectively. In another embodiment, the invention provides the compounds of Formula (III) wherein X¹, X² and X³ are single bonds. In another embodiment, the invention provides the compounds of Formula (III) wherein R⁷ and R¹⁸ are hydrogen.

The 1,2-dihydropyridine compound used in the methods and compositions described herein may be Compound A:

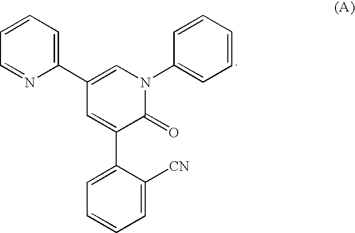

The IUPAC name for Compound A is 2-(2-oxo-1-phenyl-5-pyridin-2-yl-1,2-dihydropyridin-3-yl)benzonitrile. Compound A may also be referred to as 3-(2-cyanophenyl)-5-(2-pyridyl)-1-phenyl-1,2-dihydropyridin-2-one. Compound A is also known as perampanel.

The terms “Compound A,” “2-(2-oxo-1-phenyl-5-pyridin-2-yl-1,2-dihydropyridin-3-yl)benzonitrile,”, “3-(2-cyanophenyl)-5-(2-pyridyl)-1-phenyl-1,2-dihydropyridin-2-one” and “perampanel” are intended to include pharmaceutically acceptable salts thereof, stereoisomers thereof, pharmaceutically acceptable salts of stereoisomers thereof, hydrates thereof, hydrates of pharmaceutically acceptable salts thereof, stereoisomers of hydrates thereof, and stereoisomer of hydrates of pharmaceutically acceptable salts thereof. In another embodiment, the terms “Compound A,” “2-(2-oxo-1-phenyl-5-pyridin-2-yl-1,2-dihydropyridin-3-yl)benzonitrile,”, “3-(2-cyanophenyl)-5-(2-pyridyl)-1-phenyl-1,2-dihydropyridin-2-one” and “perampanel” are intended to include pharmaceutically acceptable salts thereof, hydrates thereof, and hydrates of pharmaceutically acceptable salts thereof.

In other embodiments, the 1,2-dihydropyridine compounds that are useful in the methods and compositions of the invention are 3-(2-cyanophenyl)-5-(2-methylsulfonylaminophenyl)-1-phenyl-1,2-dihydropyridin-2-one; 3-(2-chloro-3-pyridyl)-5-(2-pyridyl)-1-phenyl-1,2-dihydropyridin-2-one; 3-(2-cyanophenyl)-5-(2-pyridyl)-1-phenyl-1,2-dihydropyridin-2-one; 3-(2-cyanophenyl)-5-(2-pyridyl)-1-(3-nitrophenyl)-1,2-dihydropyridin-2-one; 3-(2-cyanophenyl)-5-(2-pyridyl)-1-(3-aminophenyl)-1,2-dihydropyridin-2-one; 3-(2-cyanophenyl)-5-(2-pyridyl)-1-(3-methylsulfonylaminophenyl)-1,2-dihydropyridin-2-one; 3-(2-cyanophenyl)-5-(2-pyridyl)-1-(3-methylaminophenyl)-1,2-dihydropyridin-2-one; 3-(2-cyanophenyl)-5-(2-pyridyl)-1-(3 dimethylaminophenyl)-1,2-dihydropyridin-2-one; 3-(2-cyanophenyl)-5-(2-pyridyl)-1-[3-(5-methoxymethyl-2-oxazolidinon-3-yl)-phenyl]-1,2-dihydropyridin-2-one; 3-(2-cyanophenyl)-5-(2-pyridyl)-1-(3-methoxycarbonylphenyl)-1,2-dihydropyridin-2-one; 3-(2-cyanophenyl)-5-(2-pyridyl)-1-(3-methylaminocarbonylphenyl)-1,2-dihydropyridin-2-one; 3-(2-cyano-3-pyridyl)-5-(2-pyridyl)-1-phenyl-1,2-dihydropyridin-2-one; 3-(2-chlorophenyl)-5-(2-pyridyl)-1-(4-hydroxyphenyl)-1,2-dihydropyridin-2-one; 3-(2-chlorophenyl)-5-(2-pyridyl)-1-(4-dimethylaminoethoxyphenyl)-1,2-dihydropyridin-2-one; 3-(2-cyanophenyl)-5-(2-pyridyl)-1-(3-formylphenyl)-1,2-dihydropyridin-2-one; 3-(2-cyanophenyl)-5-(2-pyridyl)-1-(3-hydroxymethylphenyl)-1,2-dihydropyridin-2-one; 3-(2-cyanophenyl)-5-(2-pyridyl)-1-(3-cyanomethylphenyl)-1,2-dihydropyridine-2-one; 3-(2-cyanophenyl)-5-(2-pyridyl)-1-(3-acetylaminomethylphenyl)-1,2-dihydropyridin-2-one; 3-(2-cyanophenyl)-5-(2-pyridyl)-1-(3-methylsulfonylaminomethylphenyl)-1,2-dihydropyridin-2-one; 3-(2-cyanophenyl)-5-(2-pyridyl)-1-(3-acetoxymethylphenyl)-1,2-dihydropyridin-2-one; 3-(2-cyanophenyl)-5-(2-pyridyl)-1-(4-methylthiophenyl)-1,2-dihydropyridin-2-one; 3-(2-cyanophenyl)-5-(2-pyridyl)-1-(4-methylsulfonylpheny-1)-1,2-dihydropyridin-2-one; 3-(2-cyanophenyl)-5-(2-formylthiophen-3-yl)-1-phenyl-1,2-dihydropyridin-2-one; 3-(2-cyanophenyl)-5-(2-diethylaminomethylthiophen-3-yl)-1-phenyl-1,2-dihydropyridin-2-one; 3-(2-cyanophenyl)-5-(2-hydroxymethylthiophen-3-yl)-1-phenyl-1,2-dihydropyridine-2-one; 3-(2-cyanophenyl)-5-(2-pyridyl)-1-benzyl-1,2-dihydropyridin-2-one; 3-(2-cyanophenyl)-5-(2-pyridyl)-1-(3-pyridyl)-1,2-dihydropyridin-2-one; 3-(2-cyanophenyl)-5-phenyl-(2-pyridyl)-1,2-dihydropyridin-2-one; 3-(2-cyanophenyl)-1,5-diphenyl-1,2-dihydropyridin-2-one; 3-(2-cyanophenyl)-5-(2-methoxyphenyl)-1-phenyl-1,2-dihydropyridin-2-one; 3-(2-cyanophenyl)-5-(3,4-dimethoxyphenyl)-1-phenyl-1,2-dihydropyridin-2-one; 3-(2-cyanophenyl)-5-(thiophen-3-yl)-1-phenyl-1,2-dihydropyridin-2-one; 3-(2-cyanophenyl)-5-(2-fluorophenyl)-1-phenyl-1,2-dihydropyridin-2-one; 3-(2-cyanophenyl)-5-(thiophen-2-yl)-1-phenyl-1,2-dihydropyridin-2-one; 3-(2-cyanophenyl)-5-(3-furfuryl)-1-phenyl-1,2-dihydropyridin-2-one; 3-(2-cyanophenyl)-5-(2-furfuryl)-1-phenyl-1,2-dihydropyridin-2-one; 3-(2-chlorophenyl)-5-(2-pyridyl)-1-phenyl-1,2-dihydropyridin-2-one; 3-(2-methoxycarbonylphenyl)-5-(2-pyridyl)-1-phenyl-1,2-dihydropyridin-2-one; 3-phenyl-5-(2-pyridyl)-1-phenyl-1,2-dihydropyridin-2-one; 3-(2-fluorophenyl)-5-(2-pyridyl)-1-phenyl-1,2-dihydropyridin-2-one; 3-(2-chlorophenyl)-5-(2-pyridyl)-1-(3-methoxyphenyl)-1,2-dihydropyridin-2-one; 3-(2-fluoro-3-pyridyl)-5-(2-pyridyl)-1-phenyl-1,2-dihydropyridin-2-one; 3-(4-methoxy-3-pyridyl)-5-(2-pyridyl)-1-phenyl-1,2-dihydropyridin-2-one; 3-(2-fluoro-3-pyridyl)-5-(2-pyridyl)-1-(3-methoxyphenyl)-1,2-dihydropyridin-2-one; 3-(2-fluoro-3-pyridyl)-5-(2-pyridyl)-1-(3-fluorophenyl)-1,2-dihydropyridin-2-one; 3-(2-cyanophenyl)-5-(2-pyridyl)-1-(4-fluorophenyl)-1,2-dihydropyridin-2-one; 3-(2-cyanophenyl)-5-(2-pyridyl)-1-(3-fluorophenyl)-1,2-dihydropyridin-2-one; 3-(2-cyanophenyl)-5-(2-pyridyl)-1-(4-methoxyphenyl)-1,2-dihydropyridin-2-one; 3-(2-cyanophenyl)-5-(2-pyridyl)-1-(3-methoxy-phenyl)-1,2-dihydropyridin-2-one; 3-phenyl-5-(2-pyridyl)-1-(3-fluorophenyl-)-1,2-dihydropyridin-2-one; 3-(2-chlorophenyl)-5-(2-pyridyl)-1-(4-fluorophenyl)-1,2-dihydropyridin-2-one; 3-(2-cyanophenyl)-5-(2-pyridyl)-1-(4-formylphenyl)-1,2-dihydropyridin-2-one; 3-(2-cyanophenyl)-5-(2-pyridyl)-1-(2-formylphenyl)-1,2-dihydropyridin-2-one; 3-(2-cyanophenyl)-5-(2-pyridyl)-1-(3-chlorophenyl)-1,2-dihydropyridin-2-one; 3-(2-cyanophenyl)-5-(2-pyridyl)-1-(3-tolyl)-1,2-dihydropyridin-2-one; 3-(2-cyanophenyl)-5-(2-pyridyl)-1-(3-trifluoromethylphenyl)-1,2-dihydropyridin-2-one; 3-(2-cyanophenyl)-5-(2-pyridyl)-1-(thiophen-3-yl)-1,2-dihydropyridin-2-one; 3-(2-cyanophenyl)-5-(2-pyridyl)-1-(3-furfuryl)-1,2-dihydropyridin-2-one; 3-(2-cyanophenyl)-5-(2-pyridyl)-1-(4-tolyl)-1,2-dihydropyridin-2-one; 3-(2-cyanophenyl)-5-(2-pyridyl)-1-(4-trifluoromethylphenyl)-1,2-dihydropyridin-2-one; 3-(2-cyanophenyl)-5-(2-pyridyl)-1-(2-methoxypyridin-5-yl)-1,2-dihydropyri-din-2-one; 3-(2-cyanophenyl)-5-(2-pyridyl)-1-(pyrimidin-5-yl)-1,2-dihydrop-yridin-2-one; 3-(2-cyanophenyl)-5-(2-pyridyl)-1-(3-benzyloxymethylpyridin-5-yl)-1,2-dihydropyridin-2-one; 3-(2-cyanophenyl)-5-(2-pyridyl)-1-(2-ethylthiopyridin-5-yl)-1,2-dihydropyridin-2-one; 3-(2-cyanophenyl)-5-(2-pyridyl-)-1-(4-pyridyl)-1,2-dihydropyridin-2-one; 3-(2-cyanophenyl)-5-(2-pyridyl)-1-(3-methoxypyridin-5-yl)-1,2-dihydropyridin-2-one; 3-(2-cyanophenyl)-5-(2-pyridyl)-1-(2-chloropyridin-5-yl)-1,2-dihydropyridin-2-one; 3-(2-cyanophenyl)-5-(2-pyridyl)-1-(2-fluoropyridin-5-yl)-1,2-dihydropyridin-2-one; 3-(2-cyanophenyl)-5-(2-pyridyl)-1-(2-methoxyphenyl)-1,2-dihydropyridin-2-one; 3-phenyl-5-(2-pyridyl)-1-(3-pyridyl)-1,2-dihydropyridin-2-one; 3-(2-chlorophenyl)-5-(2-pyridyl)-1-(3-pyridyl)-1,2-dihydropyridin-2-one; 3-(thiophen-3-yl)-5-(2-pyridyl)-1-(3-pyridyl)-1,2-dihydropyridin-2-one; 3-(2,6-dimethylphenyl)-5-(2-pyridyl)-1-(3-pyridyl)-1,2-dihydropyridin-2-one; 3-(2-cyanothiophen-3-yl)-5-(2-pyridyl)-1-(3-pyridyl)-1,2-dihydropyridin-2-one; 3-(2-fluoro-3-pyridyl)-5-(2-pyridyl)-1-(3-pyridyl)-1,2-dihydropyridin-2-one; 3-(2-chlorophenyl)-5-(2-pyridyl)-1-(3-hydroxyphenyl)-1-,2-dihydropyridin-2-one; 3-(2-chlorophenyl)-5-(2-pyridyl)-1-(3-dimethylaminoethoxyphenyl)-1,2-dihydropyridin-2-one; 3-(2 chlorophenyl)-5-(2-pyridyl)-1-(3-dimethylaminopropoxyphenyl)-1,2-dihydropyridin-2-one; 3-(2-cyanophenyl)-5-(2-pyridyl)-1-(2-hydroxymethylphenyl)-1,2-dihydropyridin-2-one; 3-(2-cyanophenyl)-5-(2-pyridyl)-1-(4-cyanomethylphenyl)-1,2-dihydropyridin-2-one; 3-(2-cyanophenyl)-5-(2-pyridyl)-1-(2-cyanomethylphenyl)-1,2-dihydropyridin-2-one; 3-(2-cyanophenyl)-5-(6-diethylaminomethyl-2-pyridyl)-1-phenyl-1,2-dihydropyridin-2-one; 3-(2-cyanophenyl)-1-phenyl-5-(2-pyrimidinyl)-1,2-dihydropyridin-2-one; 3-(2-hydroxypyridin-6-yl)-1-phenyl-5-(2-pyridyl)-1,2-dihydropyridin-2-one; 1-(2-aminobenzothiazol-6-yl)-3-(2-cyanophenyl)-5-(2-pyridyl)-1,2-dihydropyridin-2-one; 3-(2-cyanophenyl)-5-(2-pyridyl)-1-(1-benzyl-1,2,3,6-tetrahydropyridin-5-yl)-1,2-dihydropyridin-2-one; 3-[2-(5-methyl-1,2,4-oxadiazol-3-yl)phenyl]-1-phenyl-5-(2-pyridyl)-1,2-dihydropyridin-2-one; 3-(2-cyanophenyl)-5-(6-methylpyridin-2-yl)-1-phenyl-1,2-dihydropyridin-2-one; 3-(2-cyanophenyl)-5-(5-methylpyridin-2-yl)-1-phenyl-1,2-dihydropyridin-2-one; 3-(2-cyanophenyl)-5-(3-hydroxypyridin-2-yl)-1-phenyl-1,2-dihydropyridin-2-one; 3-(2-cyanophenyl)-1-phenyl-5-(2-thiazolyl)-1,2-dihydropyridin-2-one; 3-(2-cyanophenyl)-5-(2-methoxypyridin-6-yl)-1-phenyl-1,2-dihydropyridin-2-one; 1-(4-aminophenyl)-3-(2-cyanophenyl)-5-(2-pyridyl)-1,2-dihydropyridin-2-one; 1-(3-aminophenyl)-3-(2-cyanophenyl)-5-(2-pyrimidinyl)-1,2-dihydropyridin-2-one; 3-(2-cyanophenyl)-5-(2-pyridyl)-1-(2-aminotoluen-4-yl)-1-,2-dihydropyridin-2-one; 3-(2-cyanophenyl)-1-[3-(dimethylaminoethoxy)phenyl]-5-(2-pyridyl)-1,2-dihydropyridin-2-one; 3-(2-cyanophenyl)-1-[3-(piperidinoethoxy)phenyl]-5-(2-pyridyl)-1,2-dihydropyridin-2-one; 3-(2-cyanophenyl)-1-[3-(pyrrolidinoethoxy)phenyl]-5-(2-pyridyl)-1,2-dihyd-ropyridin-2-one; 3-(2-cyanophenyl)-1-[3-(diisoproylaminoethoxy)phenyl]-5-(-2-pyridyl)-1,2-dihydropyridin-2-one; 3-(2-cyanophenyl)-1-[3-(4-piperidinobutyl-1-oxy)phenyl]-5-(2-pyridyl)-1,2-dihydropyridin-2-one; 3-(2-cyanophenyl)-1-(4-nitrophenyl)-5-(2-pyridyl)-1,2-dihydropyridin-2-one; 1-phenyl-5-(2-pyridyl)-3-(2-thiazolyl)-1,2-dihydropyridin-2-one; 3-(2-cyanophenyl)-1-(3-pyridyl)-5-(2-pyrimidinyl)-1,2-dihydropyridin-2-one; 3-(2-fluoropyridin-3-yl)-1-phenyl-5-(2-pyrimidinyl)-1,2-dihydropyridin-2-one; 3-(2-cyanopyridin-3-yl)-1-phenyl-5-(2-pyrimidinyl)-1,2-dihydropyridin-2-one; 3-(2-cyanophenyl)-1-(3-nitrophenyl)-5-(2-pyrimidinyl)-1,2-dihydropyridin-2-one; 3-(2-nitrophenyl)-1-phenyl-5-(2-pyridyl)-1,2-dihydropyridin-2-one; 3-(2-formylthiophen-3-yl)-5-(2-pyridyl)-1-phenyl-1,2-dihydropyrid-in-2-one; 3-(2-cyanophenyl)-5-(2-pyridyl)-1-(2-naphthyl)-1,2-dihydropyridin-2-one; 3-(2-cyanophenyl)-5-(2-pyridyl)-1-(1-naphthyl)-1,2-dihydropyridin-2-one; 5-(2-aminopyridin-6-yl)-3-(2-cyanophenyl)-1-phenyl-1,2-dihydropyridin-2-one; 5-(2-bromopyridin-6-yl)-3-(2-cyanophenyl)-1-phenyl-1,2-dihydropyridin-2-one; 3-(2-cyanophenyl)-5-(2-morphorinopyridin-6-yl)-1-phenyl-1,2-dihydropyridin-2-one; 3-(2-cyanophenyl)-1-(3-hydroxyphenyl)-5-(2-pyridyl)-1,2-dihydroppidin-2-one; 3-(2-cyanophenyl)-1-[3-(4-piperidyloxy)]phenyl-5-(2-pyridyl)-1,2-dihydropyridin-2-one; 1-[3-(N-acetylpiperidin-4-yl-oxy)phenyl]-3-(2-cyanophenyl)-5-(2-pyridyl)-1,2-dihydropyridin-2-one; 3-(2-cyanophenyl)-1-{3-[1-(methanesulfonyl)piperidin-4-yl-oxy]phenyl}-5-(2-pyridyl)-1,2-dihydropyridin-2-one; 1-[3-(N-methylpiperidin-4-yl-oxy)pheny-1]-3-(2-cyanophenyl)-5-(2-pyridyl)-1,2-dihydropyridin-2-one; 3-(6-chloro-1H-benzimidazol-2-yl)-5-(2-pyridyl)-1-phenyl-1,2-dihydropyridin-2-one; 3-(2-cyanophenyl)-5-(2-pyridyl)-1-(2-nitrotoluen-4-yl)-1,2-dihydropyridin-2-one; 3-(2-cyanothiophen-3-yl)-5-(2-pyridyl)-1-phenyl-1,2-dihydropyridin-2-one; 3-[2-(5-oxazolyl)phenyl]-1-phenyl-5-(2-pyridyl)-1,2-dihydropyridin-2-one; 3-[2-(5-oxazolyl)thiophen-3-yl]-1-phenyl-5-(2-pyridyl)-1,2-dihydropyridin-2-one; and 3-(2-ethoxycarbonylvinylthiophen-3-yl)-5-(2-pyridyl)-1-phenyl-1,2-dihydropyridin-2-one.

The 1,2-dihydropyridine compounds and methods for making the 1,2-dihydropyridine compounds are described in U.S. Pat. No. 6,949,571, US Publication No. 2004/0023973, and PCT Publication No. WO 03/047577, WO 04/009553, WO 06/004100, WO 06/004107, WO07/072,868, and WO07/072,869, the disclosures of which are incorporated by reference herein in their entirety.

Methods for making other AMPA receptor antagonists such as quinoxalinedione aminoallylphosphonates are described, for example, in WO 2005/094797.

The NMDA receptor antagonist, which is an active ingredient” may be any known in the art. In one embodiment, the NMDA receptor antagonist is represented by Formula (IV), a pharmaceutically acceptable salt thereof, a stereoisomer thereof, or a pharmaceutically acceptable salt of a stereoisomer thereof:

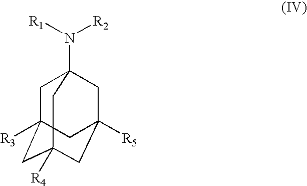

wherein R₁ and R₂ are each independently hydrogen or a straight or branched chain C₁₋₆ alkyl group, or R₁ and R₂ in conjunction with the nitrogen atom form a C₅₋₆ heterocyclic group; R₃ and R₄ are each independently hydrogen, a straight or branched chain C₁₋₆ alkyl group, a C₅₋₆ cycloalkyl group, or phenyl; and R₅ is hydrogen or a straight or branched chain C₁₋₆ alkyl group.

In one embodiment, R₁, R₂, R₃, R₄ and R₅ are not simultaneously hydrogen atoms. In one embodiment, the pharmaceutically acceptable salt is a pharmaceutically acceptable acid addition salt. Exemplary branched or straight chain C₁₋₆ alkyl groups include methyl, ethyl, iso-propyl, n-propyl, n-butyl, iso-butyl, t-butyl, n-pentyl, n-hexyl, and isomers thereof.

In one embodiment, the compound of Formula (IV) is Compound (B), a pharmaceutically acceptable salt thereof, a stereoisomer thereof, or a pharmaceutically acceptable salt of a stereoisomer thereof:

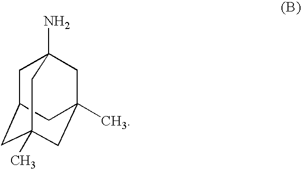

In another embodiment, the compound of Formula (IV) is Compound (B1) or a pharmaceutically acceptable salt thereof:

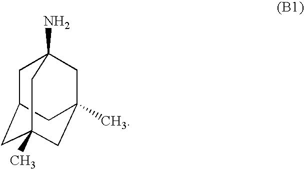

Compound (B1) is also known as memantine and can be in the form of any pharmaceutically acceptable salt known in the art. In one embodiment, memantine is in the form of a hydrochloride salt. Compounds (B) and (B1) are also known as 1-amino-3,5-dimethyl-adamantane.

In other embodiments, the adamantane compound is 1-amino adamantine; 1-amino-3-phenyl adamantine; 1-amino-methyl-adamantane; 1-amino-3,5-dimethyl adamantine; 1-amino-3-ethyl adamantine; 1-amino-3-isopropyl adamantine; 1-amino-3-n-butyl adamantine; 1-amino-3,5-diethyl adamantine; 1-amino-3,5-diisopropyl adamantine; 1-amino-3,5-di-n-butyl adamantine; 1-amino-3-methyl-5-ethyl adamantine; 1-N-methylamino-3,5-dimethyl adamantine; 1-N-ethylamino-3,5-dimethyl adamantine; 1-N-isopropyl-amino-3,5-dimethyl adamantine; 1-N,N-dimethyl-amino-3,5-dimethyl adamantine; 1-N-methyl-N-isopropyl-amino-3-methyl-5-ethyl adamantine; 1-amino-3-butyl-5-phenyl adamantine; 1-amino-3-pentyl adamantine; 1-amino-3,5-dipentyl adamantine; 1-amino-3-pentyl-5-hexyl adamantine; 1-amino-3-pentyl-5-cyclohexyl adamantine; 1-amino-3-pentyl-5-phenyl adamantine; 1-amino-3-hexyl adamantine; 1-amino-3,5-dihexyl adamantine; 1-amino-3-hexyl-5-cyclohexyl adamantine; 1-amino-3-hexyl-5-phenyl adamantine; 1-amino-3-cyclohexyl adamantine; 1-amino-3,5-dicyclohexyl adamantine; 1-amino-3-cyclohexyl-5-phenyl adamantine; 1-amino-3,5-diphenyl adamantine; 1-amino-3,5,7-trimethyl adamantine; 1-amino-3,5-dimethyl-7-ethyl adamantine; 1-amino-3,5-diethyl-7-methyl adamantine; 1-N-pyrrolidino and 1-N-piperidine derivatives; 1-amino-3-methyl-5-propyl adamantine; 1-amino-3-methyl-5-butyl adamantine; 1-amino-3-methyl-5-pentyl adamantine; 1-amino-3-methyl-5-hexyl adamantine; 1-amino-3-methyl-5-cyclohexyl adamantine; 1-amino-3-methyl-5-phenyl adamantine; 1-amino-3-ethyl-5-propyl adamantine; 1-amino-3-ethyl-5-butyl adamantine; 1-amino-3-ethyl-5-pentyl adamantine; 1-amino-3-ethyl-5-hexyl adamantine; 1-amino-3-ethyl-5-cyclohexyl adamantine; 1-amino-3-ethyl-5-phenyl adamantine; 1-amino-3-propyl-5-butyl adamantine; 1-amino-3-propyl-5-pentyl adamantine; 1-amino-3-propyl-5-hexyl adamantine; 1-amino-3-propyl-5-cyclohexyl adamantine; 1-amino-3-propyl-5-phenyl adamantine; 1-amino-3-butyl-5-pentyl adamantine; 1-amino-3-butyl-5-hexyl adamantine; 1-amino-3-butyl-5-cyclohexyl adamantine; their N-methyl, N,N-dimethyl, N-ethyl, N-propyl derivatives. Each of these compounds can be in the form of a pharmaceutically acceptable salt thereof, a stereoisomer thereof, or a pharmaceutically acceptable salt of a stereoisomer thereof.

Methods for preparing Compounds (IV), (B) and (B1) are described in U.S. Pat. No. 5,061,703, the disclosure of which is incorporated by reference herein in its entirety. Memantine is commercially available as NAMENDA® from Forest Laboratories.

In another embodiment, the NMDA receptor antagonist is a compound of Formula (V), a pharmaceutically acceptable salt thereof, a stereoisomer thereof, or a pharmaceutically acceptable salt of a stereoisomer thereof:

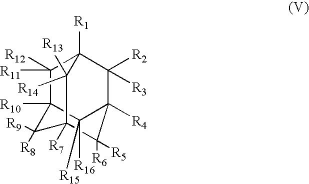

wherein R₁ is an amino group or

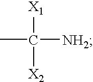

where X₁ and X₂ are each independently hydrogen or a C₁₋₅ aliphatic group; R₂ through R₁₆ are each independently hydrogen or a chain C₁₋₅ aliphatic group. R₄ and R₁₀ may alternatively and independently be halogen or an acyl group.

In one embodiment, the compound of Formula (V) is amantadine, a pharmaceutically acceptable salt thereof, a stereoisomer thereof, or a pharmaceutically acceptable salt of a stereoisomer thereof; wherein R₂-R₁₆ are all hydrogen atoms. In another embodiment, the compound of Formula (V) is memantine; a pharmaceutically acceptable salt thereof, a stereoisomer thereof, or a pharmaceutically acceptable salt of a stereoisomer thereof; memantine is shown above as Compounds (B) and (B1). In another embodiment, the compound of Formula (V) is rimantadine, a pharmaceutically acceptable salt thereof, a stereoisomer thereof, or a pharmaceutically acceptable salt of a stereoisomer thereof wherein R₂-R₁₇ are all hydrogen atoms and R₁ is

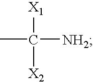

wherein X₁ is hydrogen and X₂ is methyl.

Methods for preparing Compound (V) are described in U.S. Pat. No. 5,614,560, the disclosure of which is incorporated by reference herein in its entirety.

In another embodiment, the NMDA receptor antagonist is neramexane (i.e., 1-amino-1,3,3,5,5-pentamethylcyclohexane) or a pharmaceutically acceptable salt thereof as described in U.S. Pat. No. 6,034,134 and WO 2004/037234, the disclosures of which are incorporated by reference herein in their entirety.

In other embodiments, exemplary NMDA receptor antagonists that can be used in the compositions, combinations, and methods described herein, include ketamine or a pharmaceutically acceptable salt thereof, eliprodil or a pharmaceutically acceptable salt thereof, ifenprodil or a pharmaceutically acceptable salt thereof, dizocilpine or a pharmaceutically acceptable salt thereof, remacemide or a pharmaceutically acceptable salt thereof, iamotrigine or a pharmaceutically acceptable salt thereof, riluzole or a pharmaceutically acceptable salt thereof, aptiganel or a pharmaceutically acceptable salt thereof, phencyclidine or a pharmaceutically acceptable salt thereof, flupirtine or a pharmaceutically acceptable salt thereof, celfotel or a pharmaceutically acceptable salt thereof, felbamate or a pharmaceutically acceptable salt thereof, spermine or a pharmaceutically acceptable salt thereof, spermidine or a pharmaceutically acceptable salt thereof, levemopamil or a pharmaceutically acceptable salt thereof, dextromethorphan ((+)-3-hydroxy-N-methylmorphinan) or a pharmaceutically acceptable salt thereof, dextrorphan ((+)-3-hydroxy-N-methylmorphinan) or a pharmaceutically acceptable salt thereof, and the like. These NMDA receptor antagonists are known in the art and described, for example, in PCT Publication Nos. WO 2004/071431 and WO 2006/121560, the disclosures of which are incorporated by reference herein in their entirety.

Other NMDA receptor antagonists known in the art can be used in the compositions, combinations and methods described herein, including the NMDA receptor antagonists described in U.S. Pat. Nos. 4,346,112; 5,061,703; 5,334,618; 5,382,601; 6,444,702; 6,620,845; and 6,662,845, the disclosures of which are incorporated by reference herein in their entirety.

The dosage form of the formulation included in the combination, kit and/or pharmaceutical composition of the invention is not particularly limited as long as it contains APMA receptor antagonists and NMDA receptor antagonists. The combination, kit and/or pharmaceutical composition of the invention is useful as a combination, kit and/or a pharmaceutical composition for treating neurodegenerative diseases (e.g., Alzheimer's disease, Parkinson's disease, Huntington's disease, amyotrophic lateral sclerosis, multiple sclerosis, Pick's disease, and the like), dementia, mild cognitive impairments, glaucoma, ocular hypertension, and pain (e.g., neuropathic pain, headaches, and the like).

The combination, kit and/or pharmaceutical composition of the invention may be used as a drug for treating neurodegenerative diseases (e.g., Alzheimer's disease, Parkinson's disease, Huntington's disease, amyotrophic lateral sclerosis, multiple sclerosis, Pick's disease, and the like), dementia, mild cognitive impairments, glaucoma, ocular hypertension, and pain (e.g., neuropathic pain, headaches, and the like).

The combination, kit and/or pharmaceutical composition of the invention may be administered to a patient.

The combination, kit and/or pharmaceutical composition of the invention may be used through oral or parental administration. When the combination, kit and/or pharmaceutical composition of the invention is used, the given dose of the compound of the invention differs depending on the degree of the symptom, age, sex, weight and sensitivity difference of the patient, administration mode, administration period, administration interval, nature, prescription and the type of the pharmaceutical formulation, and the type of the active element. Usually, but without limitation, the dose of the compound is 0.1 mg/day to 1000 mg/day, preferably 0.5 mg/day to 100 mg/day, more preferably 1 mg/day to 40 mg/day for an adult (weight 60 kg), which may be administered once to three times a day.

The pharmaceutical composition of the invention may be made into various forms, for example, into solid oral formulations, injectable solution or the like.

The AMPA receptor antagonists and the NMDA receptor antagonists can be administered orally, topically, parenterally, by inhalation (nasal or oral), or rectally in dosage unit formulations containing conventional nontoxic pharmaceutically acceptable excipients as desired. The term parenteral includes subcutaneous, intravenous, intramuscular, intrathecal, intrasternal injection, or infusion techniques.

The daily dose of the AMPA receptor antagonists, such as the 1,2-dihydropyridine compounds of the invention (e.g., 3-(2-cyanophenyl)-5-(2-pyridyl)-1-phenyl-1,2-dihydropyridin-2-one) is usually 30 μg/day to 10 g/day; preferably 100 μg/day to 5 g/day or, more preferably 100 μg/day to 100 mg/day in the case of oral administration. For administration by injection, the daily dose is usually 30 μg/day to 1 g/day; preferably 100 μg/day to 500 mg/day or, more preferably 100 μg/day to 30 mg/day. The AMPA receptor antagonists are administered once daily or in several portions a day. When used in the context of a dosage amount, the numerical weight refers to the weight of the AMPA receptor antagonist, such as the 1,2-dihydropyridine compounds of the invention, exclusive of any salt, counterion, hydrate, and the like. Therefore to obtain the equivalent of 500 mg of 3-(2-cyanophenyl)-5-(2-pyridyl)-1-phenyl-1,2-dihydropyridin-2-one, it would be necessary to use more than 500 mg of a pharmaceutically acceptable salt and/or hydrate of the compound, due to the additional weight of the pharmaceutically acceptable salt and/or hydrate. The daily dose of perampanel is usually 1 mg/day to 30 mg/day, preferably 1 mg/day to 20 mg/day, preferably 2 mg/day to 12 mg/day, or more preferably 2 mg/day to 8 mg/day in the case of oral administration.

The daily dose of the NMDA receptor antagonists, such as the adamantane compounds of the invention (e.g., memantine) is usually 0.01 to 100 mg/day; 0.01 mg/day to 1000 mg/day; preferably 0.1 mg/day to 500 mg/day; preferably 1 mg/day to 100 mg/day; preferably 1 mg/day to 50 mg/day; or more preferably 10 mg/day to 30 mg/day. The NMDA receptor antagonists are administered once daily or in several portions a day. When used in the context of a dosage amount, the numerical weight refers to the weight of the NMDA receptor antagonist, exclusive of any salt, counterion, hydrate, and the like. Therefore to obtain the equivalent of 500 mg of memantine, it would be necessary to use more than 500 mg of a pharmaceutically acceptable salt of memantine, due to the additional weight of the pharmaceutically acceptable salt. The daily dose of memantine is usually 1 mg/day to 40 mg/day, preferably 5 mg/day to 28 mg/day, preferably 5 mg/day to 20 mg/day, or more preferably 10 mg/day to 20 mg/day in the case of oral administration.

In one embodiment, the mode of administration is by injection, such as subcutaneous injection, intramuscular injection, intravenous injection, or intra-arterial injection. Injectable preparations, for example, sterile injectable aqueous or oleaginous suspensions can be formulated according to the art using suitable dispersing or wetting agents, suspending agents (e.g., methylcellulose, Polysorbate 80, hydroxyethylcellulose, acacia, powdered tragacanth, sodium carboxymethylcellulose, polyoxytehylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like), stabilizers (e.g., sodium sulfite and sodium metasulfite; examples of the preservative include methyl parahydroxybenzoate, ethyl parahydroxybenzoate, sorbic acid, phenol, cresol, chlorocresol, and the like), tonicity agents and preservatives. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. In addition, sterile, fixed oils are conventionally used as a solvent or suspending medium. For this purpose any bland fixed oil can be used including synthetic mono- or diglycerides, in addition, fatty acids, such as oleic acid, can be used in the preparation of injectables. The preparations can be lyophilized by methods known in the art.

In order to prepare a solid oral formulation, an excipient, and if necessary, a binder, disintegrant, lubricant, colorant, a flavoring agent and the like are added to the principal agent, and then made into a tablet, a coated tablet, granule, fine granule, dispersant, a capsule or the like according to a conventional method.

For example, lactose, cornstarch, sucrose, glucose, sorbit, crystalline cellulose, silicon dioxide or the like may be used as the excipient; for example, polyvinyl alcohol, ethyl cellulose, methyl cellulose, gum arabic, hydroxypropyl cellulose, hydroxypropylmethyl cellulose or the like may be used as the binder; for example, magnesium stearate, talc, silica or the like may be used as the lubricant; those that are allowed to be added to drugs may be used as the colorant; and for example, cocoa powder, menthol, aromatic acid, peppermint oil, camphor, cinnamon powder or the like may be used as the flavoring agent. Of course, if necessary, these tablets and granule may be coated appropriately with sugar coating, gelatin coating or else.

Solid dosage forms for oral administration can include chewing gum, capsules, tablets, sublingual tablets, powders, granules, and gels. In such solid dosage forms, the active ingredient(s) can be admixed with one or more inert diluents such as lactose or starch. As is normal practice, such dosage forms can also comprise other substances including lubricating agents such as magnesium stearate. In the case of capsules, tablets, and pills, the dosage forms can also comprise buffering agents. The tablets can be prepared with enteric or film coatings.

To make tablets, the active ingredient(s) can be admixed with pharmaceutically acceptable carriers known in the art such as vehicles (e.g., lactose, white sugar, mannitol, glucose, starches, calcium carbonate, crystalline cellulose, silicic acid, and the like), binders (e.g., water, ethanol, myranol, glucose solution, starch solution, gelatin solution, polyvinylpyrrolidone, and the like), disintegrators (e.g., dry starch, sodium, alginate, sodium hydrogen carbonate, calcium carbonate, polyoxyethylene sorbitan fatty acid esters, sodium laurylsulfate, stearic monoglyceride, starches, lactose, and the like), absorption promoters (e.g., quaternary ammonium base, sodium laurylsulfate, and the like), wetting agents (e.g. glycerin, starches, and the like), lubricants (e.g., stearates, polyethylene glycol, and the like), and flavoring agents (e.g., sweeteners). The tablets can be in the form of a conventional tablet, a molded tablet, a wafer and the like. Sublingual administration refers to the administration in the mouth (e.g., under the tongue, between the cheek and gum, between the tongue and roof of the mouth). The highly vascular mucosal lining in the mouth is a convenient location for the active ingredients to be administered into the body.

In other embodiments, the solid dosage form can be packaged as granules or a powder in a pharmaceutically acceptable carrier, where the granules or powder are removed from the packaging and sprinkled on food or mixed with a liquid, such as water or juice, or where the granules are inserted into capsules. In this embodiment, the active ingredients described herein can be mixed with flavoring or sweetening agents. The packaging material can be plastic, coated paper, or any material that prevents water or moisture from reaching the granules and/or powder.

Liquid dosage forms for oral administration can include pharmaceutically acceptable gels, emulsions, solutions, sublingual solutions, suspensions, and syrups containing inert diluents commonly used in the art, such as water. Such compositions can also comprise adjuvants, such as wetting agents, emulsifying and suspending agents, and sweetening, flavoring, and perfuming agents. To make sublingual solutions, the active ingredients can be admixed with various carriers, excipients, pH adjusters, and the like (e.g., water, sugar, lactic acid, acetic acid, fructose, glucose, saccharin, polyethylene glycol, propylene glycol, alcohol, bentonite, tragacanth, gelatin, alginates, aspartame, sorbitol, methylparaben, propylparaben, sodium benzoate, artificial flavoring and coloring agents). In other embodiments, the liquid dosage form can be an ophthalmic composition suitable for topical administration to the eye.

Hereinafter, the present invention will be illustrated by way of specific examples, although the invention should not be limited thereto.

### EXAMPLE

### Materials and Methods

### Cortical Cell Cultures

Cortical cell cultures were prepared from fetal rats of the Wistar strain (gestational age of 17 to 19 days, Charles River Japan, Kanagawa, Japan). The cortex was dissected and kept in Neurobasal medium (Invitrogen Corp., Carlsbad, Calif., USA) containing 2% B27 supplement (Invitrogen), 25 μM 2-mercaptoethanol (Wako), 0.5 mM L-glutamine (Invitrogen) and ×1/100 volume of antibiotic-antimycotic (Invitrogen), then incubated at 37° C. for 30 min in Ca²⁺/Mg²⁺ free Hank's balanced salt solution (MSS; 10 mM HEPES, pH 7.3) containing 0.25% trypsin (Invitrogen) and 0.2 mg/ml deoxyribonuclease (DNase) (Sigma). The cortical tissues were dissociated to single cells by gentle trituration. The cell suspension was centrifuged at 310×g for 3 min and the resulting pellets were resuspended in the medium described above.

### NMDA and Kainate-Induced Toxicity

The cells were plated at an initial cell density of 5×10⁵ cells/cm² in poly-L-lysine (Sigma)-coated 96-well plates. The cells were cultured in a CO₂ incubator (5% (v/v), at 37° C. in the Neurobasal medium described above for 7 days, and cultured following 4 days in culture medium without antioxidant B27 supplement and 2-mercaptoethanol. The cultures were exposed to 100 μM of N-methyl-D-aspartate (NMDA)(Sigma) and 100 μM of kainate (Sigma) for 24 h at 37° C. The cells were treated with perampanel (0.03, 0.3, 3 or 30 μM) and memantine HCl (0.1, 1, 10 or 100 μM) for 24 h before and during the exposure to these agonists. In the combination experiment, perampanel at 3 mM or memantine at 10 μM or its combination was used.

### MTT Assay

Viability of cells grown in 96-well plates was measured by 3-[4,5-dimethylthylthiazol-2-yl]-2,5-diphenyltetrazolium bromide (MTT) assay. The 10 μl of 8 mg/ml MTT (Sigma) solution in D-PBS(−) (Dulbecco's phosphate-buffered saline, Sigma) was added to each well. During 10 min incubation at 37° C., the active dehydrogenase in viable mitochondria reduces the tetrazolium ring of MTT, forming a blue-colored precipitate. The cells were lysed and the precipitate was dissolved in 100 ml of MTT solubilizing buffer containing 20% sodium dodecyl sulfate (SDS: Wako) in 50% N,N-dimethyl formamide at pH 4.7, and quantified spectrophotometrically at 550-690 nm.

Wells without cells, containing only the medium and MTT solution, were measured to obtain the background value, which was subtracted from measured values at 550-690 nm, as follows: % of Recovery=(sample−NMDA/Kainate treated control)/(non-treated control−NMDA/Kainate treated control)×100.

### Statistical Analysis

The data were analyzed with one-way analysis of variance (ANOVA) followed by Dunnett's multiple comparison test using the software package SAS ver. 8.2 (SAS Institute Japan Ltd., Tokyo, Japan).

### Results

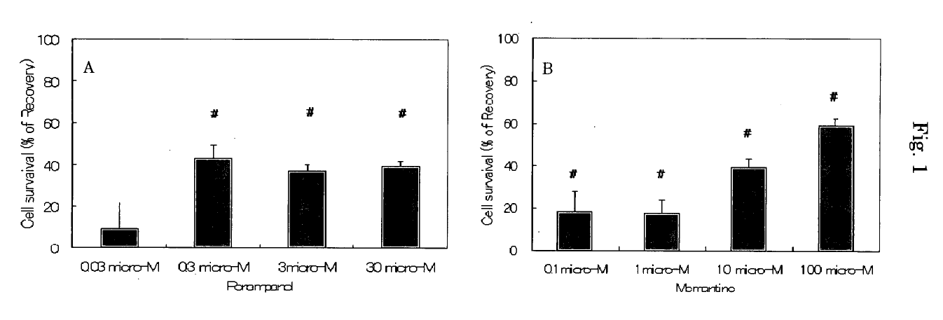

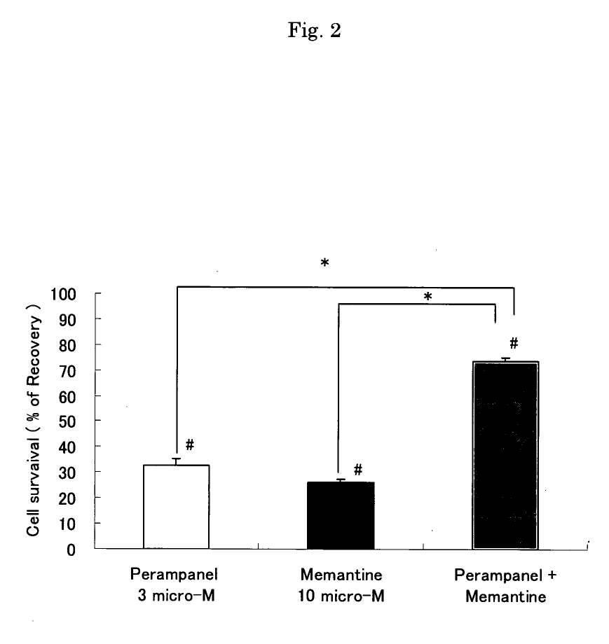

Perampanel and memantine protected neuron from excitotoxicity induced by combination treatment with kainate and NMDA. (FIG. 1A, B) Combination of perampanel and memantine caused over additive effect in this assay. (FIG. 2)

FIG. 1:

A, Effect of perampanel on neuronal damage induced by combination of kainate and NMDA. B, Effect of memantine on neuronal damage induced by combination of kainate and NMDA. #, control vs drug. P<0.05 by Dunnett's multiple comparison test.

FIG. 2:

Effect of perampanel, memantine alone and combination therapy on neuronal injury-induced by combination of kainate and NMDA. #, control vs drug, P<0.05 Dunnett's multiple comparison test, *, drug alone vs combination therapy, P<0.05.

The efficacy of memantine in a variety of dementias has been shown in clinical trials. Memantine is considered to be a promising neuroprotective drug for the treatment of dementias, particularly Alzheimer's disease (Jain K K, Evaluation of memantine for neuroprotection in dementia. Expert Opin Investig Drugs 2000; 9(6): 1397-406.). Clinical dosage of mamentine is 20 mg/day. Maximal plasma concentration of memantine at 20 mg was at around 20-25 ng/ml after single dose treatment (Periclou A, et al., Pharmacokinetic study of memantine in healthy and renally impaired subjects. Clin Pharmacol Ther 2006; 79: 134-43.). This plasma concentration roughly correspond to 0.1 μM. Clinically relevant concentration of memantine could produce significant neuroprotective effect in our system but effect size of memantine at clinically relevant concentration was small and far below maximal effect.

Perampanel showed significant neuroprotective effect from 0.3 to 30 μM in same assay system but it showed ceiling on the effect from lowest concentration showing neuroprotection.

Thus both compounds can protect neuron from the damage but monotherapy with both compounds may not cause maximal effect. In fact it was reported that clinical effect of memantine is modest in Alzheimer's disease trials. Dose increment may be a possibility of improvement of clinical efficacy however it is known that mementine at 40 mg/day showed higher risk of adverse event compared to dose at 20 mg/day (Center for Drug Evaluation and Research Approval Package for: Application Number 21-487, Medical Review Part 3, http://www.fda.gov/cder/foi/nda/2003/21-487_Namenda_Medr_P3.pdf).

Here we showed that combination of memantine and perampanel can cause over additive effect in neuronal protection. Combination therapy with memantine and perampanel could overcome current situation of clinical practice.

Each of the patents, patent applications, and publications cited herein are incorporated by reference herein in their entirety.

It will be apparent to one skilled in the art that various modifications can be made to the invention without departing from the spirit or scope of the appended claims.

1. A pharmaceutical composition comprising:
(i) perampanel, a pharmaceutically acceptable salt thereof, a hydrate thereof, or a hydrate of a pharmaceutically acceptable salt thereof;
(ii) memantine or a pharmaceutically acceptable salt thereof; and
(iii) one or more pharmaceutically acceptable carriers.

2. The pharmaceutical composition of claim 1, wherein the pharmaceutical composition is used for treating (a) a neurodegenerative disease; (b) dementia; (c) a mild cognitive impairment; (d) glaucoma; (e) ocular hypertension; or (f) pain.

3. A combination comprising:
(i) perampanel, a pharmaceutically acceptable salt thereof, a hydrate thereof, or a hydrate of a pharmaceutically acceptable salt thereof; and
(ii) memantine or a pharmaceutically acceptable salt thereof.

4. The combination of claim 3, wherein the combination is administered separately to the patient or is administered to the patient in the form of a pharmaceutical composition.

5. The combination of claim 3, wherein the combination is used for treating (a) a neurodegenerative disease; (b) dementia; (c) a mild cognitive impairment; (d) glaucoma; (e) ocular hypertension; or (f) pain.

6. A kit comprising:
(i) perampanel, a pharmaceutically acceptable salt thereof, a hydrate thereof, or a hydrate of a pharmaceutically acceptable salt thereof,
(ii) memantine or a pharmaceutically acceptable salt thereof; and
(iii) instructions for simultaneous, separate or sequential use thereof for the treatment and/or prophylaxis of (a) a neurodegenerative disease; (b) dementia; (c) a mild cognitive impairment; (d) glaucoma; (e) ocular hypertension; or (f) pain, or for delaying the onset of (a) a neurodegenerative disease; (b) dementia; (c) a mild cognitive impairment; (d) glaucoma; (e) ocular hypertension; or (f) pain.

7. The kit of claim 6, wherein (i) perampanel, a pharmaceutically acceptable salt thereof, a hydrate thereof, or a hydrate of a pharmaceutically acceptable salt thereof; and (ii) memantine or a pharmaceutically acceptable salt thereof are administered separately to the patient or are administered to the patient in the form of a pharmaceutical composition.

8. Use of:
(i) perampanel, a pharmaceutically acceptable salt thereof, a hydrate thereof, or a hydrate of a pharmaceutically acceptable salt thereof; and
(ii) memantine or a pharmaceutically acceptable salt thereof for producing a pharmaceutical composition in the treatment of (a) a neurodegenerative disease; (b) dementia; (c) a mild cognitive impairment; (d) glaucoma; (e) ocular hypertension; or (f) pain.

9. The use of claim 8, wherein (i) perampanel, a pharmaceutically acceptable salt thereof, a hydrate thereof, or a hydrate of a pharmaceutically acceptable salt thereof; and (ii) memantine or a pharmaceutically acceptable salt thereof are administered separately to the patient or are administered to the patient in the form of a pharmaceutical composition.

10. A method for treating (a) a neurodegenerative disease; (b) dementia; (c) a mild cognitive impairment; (d) glaucoma; (e) ocular hypertension; or (0 pain, in a patient in need thereof comprising administering (i) perampanel, a pharmaceutically acceptable salt thereof, a hydrate thereof, or a hydrate of a pharmaceutically acceptable salt thereof; and (ii) memantine or a pharmaceutically acceptable salt thereof.

11. The method of claim 10, wherein (i) perampanel, a pharmaceutically acceptable salt thereof, a hydrate thereof, or a hydrate of a pharmaceutically acceptable salt thereof; and (ii) memantine or a pharmaceutically acceptable salt thereof are administered separately to the patient or are administered to the patient in the form of a pharmaceutical composition.

12. (i) A perampanel, a pharmaceutically acceptable salt thereof, a hydrate thereof, or a hydrate of a pharmaceutically acceptable salt thereof; and (ii) memantine or a pharmaceutically acceptable salt thereof in the treatment of (a) a neurodegenerative disease; (b) dementia; (c) a mild cognitive impairment; (d) glaucoma; (e) ocular hypertension; or (f) pain.
